(19) United States
(12) Patent Application Publication
(10) Pub. No.: US 2025/0314623 A1
(43) Pub. Date: Oct. 9, 2025

(54) METHODS AND SYSTEMS FOR PHOTOACOUSTIC VISUAL SERVOING

(71) Applicant: THE JOHNS HOPKINS UNIVERSITY, Baltimore, MD (US)

(72) Inventors: Muyinatu BELL, Baltimore, MD (US); Mardava GUBBI, Baltimore, MD (US)

(21) Appl. No.: 18/866,673

(22) PCT Filed: May 26, 2023
(86) PCT No.: PCT/US2023/023695

§ 371 Date: Nov. 18, 2024

Related U.S. Application Data
(60) Provisional application No. 63/346,855, filed on May 28, 2022.

(51) Int. Cl. G01N 29/22; G01N 29/06; G01N 29/24; G01N 29/44

(52) U.S. Cl. CPC G01N 29/225; G01N 29/0672; G01N 29/2418; G01N 29/4481; G01N 2291/02475

(57) ABSTRACT

Provided herein are methods of tracking the positions of medical devices using photoacoustic visual servoing. Additional methods as well as related systems and computer readable media are also provided.

### CROSS-REFERENCE TO RELATED APPLICATIONS

This application is the national stage entry of International Patent Application No. PCT/US2023/023695, filed on May 26, 2023, and published as WO 2023/235250 A1 on Dec. 7, 2023, which claims the benefit of U.S. Provisional Patent Application Ser. No. 63/346,855, filed on May 28, 2022, which are hereby incorporated by reference in their entireties.

### STATEMENT OF GOVERNMENT SUPPORT

This invention was made with government support under R21 EB025621 awarded by the National Institutes of Health and under NSF ECCS-1751522 and U.S. Pat. No. 2,014,088 awarded by the National Science Foundation. The government has certain rights in the invention.

### BACKGROUND

The integration of computer vision with medical imaging is an important subfield of modern healthcare. The dual tasks of visualizing and tracking needle tips, catheter tips, and other surgical tool tips form a significant component of numerous surgical and interventional procedures, such as percutaneous biopsies. Ultrasound imaging is commonly used for this task due to its low cost, high frame rates, portability, and the absence of harmful ionizing radiation associated with other imaging modalities such as fluoroscopy. However, ultrasound fails in imaging environments characterized by acoustic clutter, sound scattering, and signal attenuation. These limitations may be overcome by replacing the acoustic transmission component of an ultrasound imaging system with optical energy transmission to create a photoacoustic imaging system, then further integrating deep learning to overcome computer vision challenges.

### SUMMARY

In one aspect, the present disclosure relates to a system that includes an electromagnetic radiation source configured to produce electromagnetic waves, and an optical fiber operably connected to the electromagnetic radiation source, which optical fiber is configured to transmit the electromagnetic waves from the electromagnetic radiation source to one or more selected sites in and/or on a subject to generate acoustic waves at least proximal to the one or more selected sites in and/or on the subject. The system also includes a medical device operably connected to the optical fiber, and a robotic device comprising an acoustic sensor, which robotic device is configured to position the acoustic sensor to receive the acoustic waves. In addition, the system also includes a controller operably connected at least to the robotic device, which controller comprises a processor and a memory communicatively coupled to the processor, which memory stores instructions which, when executed on the processor, perform operations comprising: positioning the acoustic sensor within sensory communication of the acoustic waves using the robotic device such that the acoustic sensor receives the acoustic waves to produce image data; and tracking a position of the medical device using the image data and one or more photoacoustic point source localization algorithms selected from the group consisting of: an amplitude-based algorithm, a coherence-based algorithm, and a deep learning-based target segmentation algorithm.

In some embodiments, the instructions comprise an electronic neural network and wherein the deep learning-based target segmentation algorithm is implemented using an electronic neural network. In some embodiments, the instructions which, when executed on the processor, perform tracking the position of the medical device using the image data and two or more of the photoacoustic point source localization algorithms. In some embodiments, the medical device comprises a needle, a catheter, or a surgical implement. In some embodiments, the image data comprises beamformed image data and/or raw channel data. In some embodiments, the subject comprises a human subject.

In another aspect, the present disclosure provides a method of tracking a position of a medical device. The method includes moving an optical fiber that is operably connected to a medical device to one or more selected sites in and/or on a subject, which optical fiber is operably connected to an electromagnetic radiation source, and transmitting electromagnetic waves from the electromagnetic radiation source to the one or more selected sites in and/or on the subject through the optical fiber to generate acoustic waves at least proximal to the one or more selected sites in and/or on the subject. The method also includes positioning an acoustic sensor within sensory communication of the acoustic waves using a robotic device that is operably connected to the acoustic sensor such that the acoustic sensor receives the acoustic waves to produce image data, and tracking a position of the medical device using the image data and one or more photoacoustic point source localization algorithms selected from the group consisting of: an amplitude-based algorithm, a coherence-based algorithm, and a deep learning-based target segmentation algorithm.

In some embodiments, the deep learning-based target segmentation algorithm is implemented using an electronic neural network. In some embodiments, the method includes tracking the position of the medical device using the image data and two or more of the photoacoustic point source localization algorithms. In some embodiments, the medical device comprises a needle, a catheter, or a surgical implement. In some embodiments, the image data comprises beamformed image data and/or raw channel data. In some embodiments, the method includes tracking the position of the medical device in substantially real-time. In some embodiments, the subject comprises a human subject.

In another aspect, the present disclosure provides a computer readable media comprising non-transitory computer executable instruction which, when executed by at least electronic processor, perform at least: moving an optical fiber that is operably connected to a medical device to one or more selected sites in and/or on a subject, which optical fiber is operably connected to an electromagnetic radiation source; transmitting electromagnetic waves from the electromagnetic radiation source to the one or more selected sites in and/or on the subject through the optical fiber to generate acoustic waves at least proximal to the one or more selected sites in and/or on the subject; positioning an acoustic sensor within sensory communication of the acoustic waves using a robotic device that is operably connected to the acoustic sensor such that the acoustic sensor receives the acoustic waves to produce image data; and, tracking a position of the medical device using the image data and one or more photoacoustic point source localization algorithms selected from the group consisting of: an amplitude-based algorithm, a coherence-based algorithm, and a deep learning-based target segmentation algorithm.

In some embodiments, the deep learning-based target segmentation algorithm is implemented using an electronic neural network. In some embodiments, the non-transitory computer-executable instructions which, when executed by the electronic processor, further perform at least: tracking the position of the medical device using the image data and two or more of the photoacoustic point source localization algorithms. In some embodiments, the medical device comprises a needle, a catheter, or a surgical implement. In some embodiments, the image data comprises beamformed image data and/or raw channel data. In some embodiments, the non-transitory computer-executable instructions which, when executed by the electronic processor, further perform at least: tracking the position of the medical device in substantially real-time. In some embodiments, the subject comprises a human subject.

### BRIEF DESCRIPTION OF DRAWINGS

FIG. 1 is a flow chart that schematically shows exemplary method steps of tracking a position of a medical device according to some aspects disclosed herein.

FIG. 2 is a schematic diagram of an exemplary system suitable for use with certain aspects disclosed herein.

FIG. 3 is a block diagram illustrating the photoacoustic visual servoing system. Process A is our previously introduced segmentation-based approach to visual servoing beamformed photoacoustic signals, after the acquisition of raw photoacoustic sensor data (also known as channel data). Process B is our newly introduced deep learning-based approach to visual servoing raw photoacoustic channel data. In each case, the red rectangular overlay indicates position coordinates that are input to the robot controller.

FIG. 4 is a finite state machine component of visual servoing system (illustrated with validity checks, d(n), corresponding to Process A).

FIGS. 5A and 5B show examples of (a) acquired experimental data and (b) simulated images with reverberations directly under the source. The reverberations are observed up to 5 mm deeper than the source, and laterally centered underneath the source.

FIG. 6 is a photograph of the setup for needle tracking and probe centering experiments.

FIG. 7 is a plot of mean needle tip tracking errors as functions of the lateral shift for Processes A and B in the phantom and ex vivo tissue. The black error bars represent the standard deviation of each set of measured errors.

FIG. 8 is a directed graph illustrating relationships among the system parameters, signal power distributions, image quality metrics, and performance of computer vision tasks. The solid black lines denote relationships reported in this example. The solid gray lines denote relationships that can be mathematically derived, yet lie outside the scope of this article. The dashed gray lines denote empirically observed relationships that confound direct mathematical descriptions. k=shape of target power distribution; θ=scale of target power distribution; μ=mean of exponentially distributed background power.

FIG. 9 is a plot of target and background power distributions extracted from a photoacoustic image, with dashed lines showing two points of intersection. The shaded regions denote the overlap between the two distributions, which is used to compute the gCNR.

FIG. 10 is a plot of target and background power distributions extracted from a photoacoustic image after applying a 0.5 threshold, with black dashed lines showing the decision boundaries of the optimal classifier for the underlying image and shaded regions denoting the overlap between the two distributions. The solid lines ending in circles at x=0 denote the Dirac delta function.

FIGS. 11A-11E are photographs with Experimental setups showing various imaging environments investigated throughout this manuscript: (a) 5-mm-diameter optical fiber bundle immersed in water bath and imaged with an Alpinion L3-8 ultrasound probe, (b) catheter equipped with optical fiber inserted into in vivo swine and imaged with an Alpinion SP1-5 probe, (c) 2-mm diameter optical fiber bundle inserted into black plastisol phantom and imaged with the SP1-5 probe, (d) 2-mm-diameter optical fiber bundle inserted into ex vivo caprine heart and imaged with the SP1-5 probe, and (e) 1-mm-diameter optical fiber inserted into plastisol phantom and imaged with the L3-8 probe.

FIG. 12 is a plot of mean±one standard deviation of u as a function of channel SNR measured from photoacoustic images of a simulated 6-mm-diameter target, an experimental setup of a 5-mm-diameter optical fiber bundle in a water bath, and a 2.5-mm-diameter catheter in an in vivo porcine heart.

FIGS. 13A and 13B are plots of mean±one standard deviation of (a) e and (b) k as functions of laser energy measured from photoacoustic images acquired with a 2-mm-diameter optical fiber bundle and a plastisol phantom or ex vivo caprine heart. The maximum energy in these plots was limited to focus on the increase in k from 0.07 to 7 μJ, rather than the saturation of similar k values observed at higher laser energies.

FIG. 14 is a plot of Possible gCNR values as a function of target scale parameter θ for independent selections of target shape parameter k and mean back-ground power μ. The upper and lower bounds of gCNR are separated into Regions 1 and 2 and Regions 3-7, respectively, denoted by the circled numbers on the plot.

FIGS. 15A-151 are photoacoustic images of (a)-(c) a 6-mm-diameter simulated photoacoustic target, (d)-(f) a 5-mm-diameter optical fiber bundle in a water bath, and (g)-(i) a 2.5-mm-diameter catheter in an in vivo porcine heart at channel SNR values of (a), (d), (g) −10 dB, (b), (e), and (h) 0 dB, and (c), (f), and (i) 10 dB, respectively. The circles in each image denote the target and background ROIs. The measured gCNR value is printed at the bottom of each image.

FIGS. 16A-16C are plots of predicted and measured gCNR as functions of channel SNR computed on subsets of 810 photoacoustic images each of (a) a 6-mm-diameter simulated target, (b) a 5-mm-diameter optical fiber bundle in a water bath, and (c) a 2.5-mm-diameter catheter in an in vivo porcine heart. The black lines and gray shaded regions denote the mean±one standard deviation of our theoretical gCNR predictions. The dots and error bars denote the mean±one standard deviation of the gCNR measured using the procedure described herein.

FIGS. 17A-17C show DAS beamformed photoacoustic images of a 2-mm-diameter optical fiber bundle in (a) a plastisol phantom and (b) an ex vivo caprine heart, each acquired with a laser energy of 2.9 UJ. The target and background ROIs are marked by circles. (c) Predicted and measured gCNR as functions of laser energy, with the maximum energy limited to 7 UJ to focus on the increase at lower energies, rather than the asymptote at higher energies. The black lines and gray shaded regions denote the mean±one standard deviation of our theoretical gCNR predictions. The dots and error bars denote the mean±one standard deviation of the gCNR measurements in the phantom and ex vivo environments.

FIGS. 18A-18C are plots showing segmentation accuracy as a function of (a) gCNR, (b) SNR, and (c) CNR measured from images acquired using a 1-mm-diameter optical fiber in a plastisol phantom and in vivo tissue. The sigmoid fits shown in each plot were created using the phantom data.

FIGS. 19A-19D are plots showing Minimum and maximum values of (a) gCNR, (b) SNR, and (c) CNR measurements as functions of the threshold to applied to photoacoustic images of a 1-mm-diameter optical fiber in phantom and in vivo tissue. (d) Percentage of finite values within these measurements.

FIG. 20 is a plot showing comparison of photoacoustic gCNR values measured using the histogram-based approach with the number of bins either fixed at 256 or selected using a data-based method. The solid black line denotes the ideal case of gCNR measurements matching predicted values.

FIG. 21 is a plot showing Predicted gCNR values using the ultrasound-based gCNR framework and our photoacoustic-based gCNR prediction framework, plotted as functions of measured gCNR values (with data-based histogram bin widths) for the five datasets described herein. The solid black line denotes the ideal case of gCNR predictions matching measured values.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Classifier: As used herein, “classifier” generally refers to algorithm computer code that receives, as input, test data and produces, as output, a classification of the input data as belonging to one or another class.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” or “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., peptide sequence and binding value pair data sets or the like).

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial or electronic neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties.

System: As used herein, “system” in the context of analytical instrumentation refers a group of objects and/or devices that form a network for performing a desired objective.

### DETAILED DESCRIPTION

The present disclosure provides photoacoustic visual servoing methods, systems, and related aspects for tracking the positions of various types of medical devices as medical procedures are performed using, for example, beamformed image data and/or raw channel data.

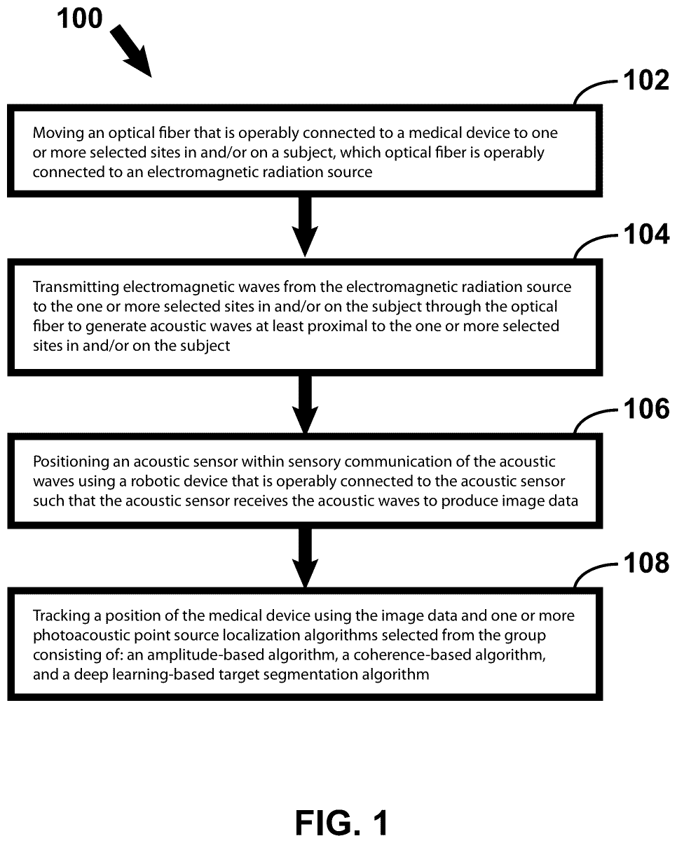

By way of overview, FIG. 1 is a flow chart that schematically shows exemplary method steps of tracking a position of a medical device according to some aspects disclosed herein. As shown, method 100 includes moving an optical fiber that is operably connected to a medical device to one or more selected sites in and/or on a subject, which optical fiber is operably connected to an electromagnetic radiation source (step 102) and transmitting electromagnetic waves from the electromagnetic radiation source to the one or more selected sites in and/or on the subject through the optical fiber to generate acoustic waves at least proximal to the one or more selected sites in and/or on the subject (step 104). Method 100 also includes positioning an acoustic sensor within sensory communication of the acoustic waves using a robotic device that is operably connected to the acoustic sensor such that the acoustic sensor receives the acoustic waves to produce image data (step 106). In addition, method 100 also includes tracking a position of the medical device using the image data and one or more photoacoustic point source localization algorithms selected from the group consisting of: an amplitude-based algorithm, a coherence-based algorithm, and a deep learning-based target segmentation algorithm (step 106).

In some embodiments, the deep learning-based target segmentation algorithm is implemented using an electronic neural network. In some embodiments, the method includes tracking the position of the medical device using the image data and two or more of the photoacoustic point source localization algorithms. In some embodiments, the medical device comprises a needle, a catheter, or a surgical implement. In some embodiments, the image data comprises beamformed image data and/or raw channel data. In some embodiments, the method includes tracking the position of the medical device in substantially real-time. In some embodiments, the subject comprises a human subject.

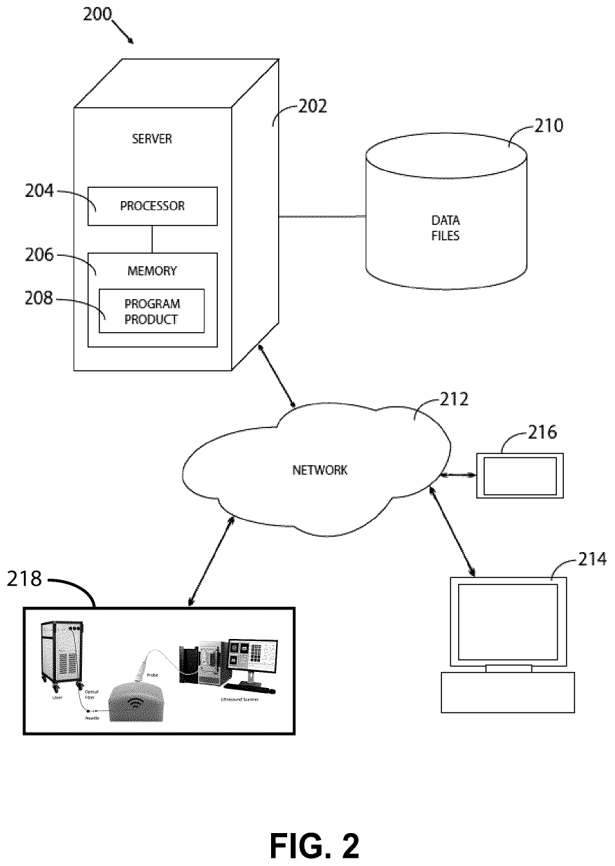

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 2 provides a schematic diagram of an exemplary system suitable for use with implementing at least aspects of the methods disclosed in this application. As shown, system 200 includes at least one controller or computer, e.g., server 202 (e.g., a search engine server), which includes processor 204 and memory, storage device, or memory component 206, and one or more other communication devices 214, 216, (e.g., client-side computer terminals, telephones, tablets, laptops, other mobile devices, etc. (e.g., for receiving molecular interaction data sets or results, etc.) in communication with the remote server 202, through electronic communication network 212, such as the Internet or other internetwork. Communication devices 214, 216 typically include an electronic display (e.g., an internet enabled computer or the like) in communication with, e.g., server 202 computer over network 212 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 200 also includes program product 208 (e.g., for tracking a position of a medical device as described herein) stored on a computer or machine readable medium, such as, for example, one or more of various types of memory, such as memory 206 of server 202, that is readable by the server 202, to facilitate, for example, a guided search application or other executable by one or more other communication devices, such as 214 (schematically shown as a desktop or personal computer). In some aspects, system 200 optionally also includes at least one database server, such as, for example, server 210 associated with an online website having data stored thereon (e.g., entries corresponding to data sets, etc.) searchable either directly or through search engine server 202. System 200 optionally also includes one or more other servers positioned remotely from server 202, each of which are optionally associated with one or more database servers 210 located remotely or located local to each of the other servers. The other servers can beneficially provide service to geographically remote users and enhance geographically distributed operations.

As understood by those of ordinary skill in the art, memory 206 of the server 202 optionally includes volatile and/or nonvolatile memory including, for example, RAM, ROM, and magnetic or optical disks, among others. It is also understood by those of ordinary skill in the art that although illustrated as a single server, the illustrated configuration of server 202 is given only by way of example and that other types of servers or computers configured according to various other methodologies or architectures can also be used. Server 202 shown schematically in FIG. 2, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 200. As also understood by those of ordinary skill in the art, other user communication devices 214, 216 in these aspects, for example, can be a laptop, desktop, tablet, personal digital assistant (PDA), cell phone, server, or other types of computers. As known and understood by those of ordinary skill in the art, network 212 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 208 is optionally in the form of microcode, programs, cloud computing format, routines, and/or symbolic languages that provide one or more sets of ordered operations that control the functioning of the hardware and direct its operation. Program product 208, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 208 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 208 is optionally copied from the computer-readable medium to a hard disk or a similar intermediate storage medium. When program product 208, or portions thereof, are to be run, it is optionally loaded from their distribution medium, their intermediate storage medium, or the like into the execution memory of one or more computers, configuring the computer(s) to act in accordance with the functionality or method of various aspects disclosed herein. All such operations are well known to those of ordinary skill in the art of, for example, computer systems.

In some aspects, program product 208 includes non-transitory computer-executable instructions which, when executed by electronic processor 204, perform at least: moving an optical fiber that is operably connected to a medical device to one or more selected sites in and/or on a subject, which optical fiber is operably connected to an electromagnetic radiation source; transmitting electromagnetic waves from the electromagnetic radiation source to the one or more selected sites in and/or on the subject through the optical fiber to generate acoustic waves at least proximal to the one or more selected sites in and/or on the subject; positioning an acoustic sensor within sensory communication of the acoustic waves using a robotic device that is operably connected to the acoustic sensor such that the acoustic sensor receives the acoustic waves to produce image data; and, tracking a position of the medical device using the image data and one or more photoacoustic point source localization algorithms selected from the group consisting of: an amplitude-based algorithm, a coherence-based algorithm, and a deep learning-based target segmentation algorithm.

As also shown in this exemplary embodiment, system 200 also includes additional system components 218, including a laser, an optical fiber, a needle, a probe, and an ultrasound scanner.

### EXAMPLES

### Example 1: Deep Learning-Based Photoacoustic Visual Servoing: Using Outputs from Raw Sensor Data as Inputs to a Robot Controller

### I. Introduction

The ability to visualize and track surgical tool tips is paramount to the success of multiple surgeries and procedures. Ultrasound is the one of the most commonly used imaging modalities to track tool tips due to its low cost, high frame rates, portability, and absence of harmful ionizing radiation. The combination of ultrasound imaging with either traditional techniques of visual servoing or recent advances in deep learning introduces additional layers of automation for this important task. For example, ultrasound-based visual servoing may assist with percutaneous needle insertions, and deep learning has the potential to improve the performance and speed of ultrasound image-based needle detection systems. However, both of these automation gains rely on the ultrasound imaging process, which tends to fail in acoustically challenging environments characterized by significant acoustic clutter, sound scattering, and sound attenuation. Specific examples of challenging acoustic environments include transcranial imaging, abdominal imaging, spinal imaging, or imaging of obese patients.

One option to address known limitations with ultrasound imaging is to combine ultrasound imaging systems with a miniature laser system to perform intraoperative photoacoustic imaging, which has provided clear images of needle tips and other structures when ultrasound imaging fails. Unlike ultrasound imaging, which requires the transmission and reception of sound to make images, photoacoustic imaging is implemented by transmitting light to generate an acoustic response that is received by the same ultrasound detectors used for ultrasound imaging. Photoacoustic imaging tends to be advantageous over ultrasound imaging in acoustically challenging environments because it only requires one way (as opposed to round-trip) acoustic travel from the transmission source to the ultrasound receiver.

Previous work from our group demonstrated the success of using photoacoustic imaging as the computer vision component of a visual servoing system, enabling continuous monitoring of needle and catheter tips. The needle or catheter tip each housed an internal optical fiber as one of the key enabling modifications to the interventional setup. This optical fiber can potentially be coupled with any surgical tool tip to enable photoacoustic-based visual servoing of the tool tip. Therefore, this approach was also demonstrated with a fiber that was independent of any tool, catheter, or needle tip.

To achieve photoacoustic-based visual servoing, raw data is typically beamformed to present a photoacoustic image that is interpretable to the human eye, followed by image segmentation to determine coordinates of interest for robot path planning. However, beamforming and other image formation approaches rely on mathematical models that do not consider all possible photoacoustic image artifact sources. Artifacts that cannot be removed with traditional amplitude-based or coherence-based photoacoustic visual servoing approaches (e.g., reflection artifacts or coherent artifacts, respectively) are confusing for both human and robot interpretation, resulting in unreliable segmentation for photoacoustic visual servoing tasks.

In order to better discriminate sources from artifacts, we turn our attention to investigate novel input sources to the robotic system (which may not necessarily need to operate on an image that is interpretable to humans). In particular, our recent photoacoustic-based deep learning approaches for photoacoustic source detection suggest that deep learning is a viable solution to address current challenges with amplitude- or coherence-based photoacoustic visual servoing. The novel concept of using deep learning to detect interventional structures of interest in raw sensor data before the application of traditional image formation techniques was previously implemented to detect needle and catheter tips. In summary, recent work from our group independently demonstrated two key advances with regard to interventional tool tip tracking: (1) photoacoustic-based visual servoing to enhance tool tip tracking and centering within the image plane and (2) deep learning-based photoacoustic image formation from raw sensor data to improve tool tip visibility.

The independent demonstrations of feasibility described above suggest that the integration of deep learning with photoacoustic-based visual servoing is a superior approach to address well-known challenges with tool tip tracking. This paper presents the first known deep learning-based photoacoustic visual servoing system to address these challenges. The novelty of this contribution includes the creation and implementation of a direct pathway from the photoacoustic raw sensor data (i.e., before any image has been formed) to the robot controller, enabled by recent advances using deep learning to extract information directly from raw acoustic sensor data.

The remainder of this example is organized as follows. Section II introduces our deep learning-based approach to visual servoing raw photoacoustic sensor data (also known as channel data), followed by a description of our network training process. This deep learning approach is contrasted with our previously introduced segmentation-based approach to visual servoing beamformed photoacoustic signals. Section III describes our experiments to test both approaches. Section IV presents our experimental results. Section V discusses our findings in the context of prior work.

### II. Visual Servoing System

### A. System Components

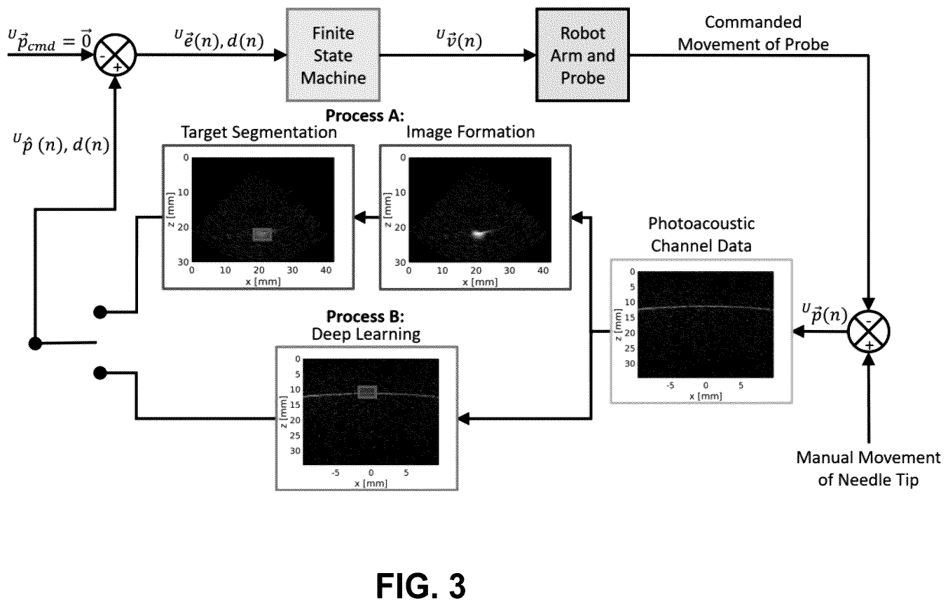

FIG. 3 shows a block diagram of the photoacoustic visual servoing system used in this work. The system components include a Sawyer robot (Rethink Robotics, Boston, MA, USA), a Vantage 128 ultrasound scanner (Verasonics Inc., Kirkland, WA, USA), a Verasonics P4-2v phased array ultrasound probe, a Phocus Mobile laser (Opotek, Carlsbad, CA, USA), and a 600 μm core diameter optical fiber. One end of the optical fiber was coupled to the laser. The other end of the optical fiber was inserted into a hollow core needle, ensuring coincident fiber and needle tips to form a fiber-needle pair. The probe was attached to the end effector of the robot using a 3D-printed holder. Nanosecond laser pulses were transmitted at a rate of 10 Hz with a wavelength of 750 nm. The software components of the visual servoing system were implemented using the Robot Operating System (ROS).

The frame U was assigned to coincide with the Verasonics P4-2v probe, with the x-, y-, and z-dimensions corresponding to the lateral, elevation, and axial dimensions of the probe, respectively. The imaging plane of the probe corresponded to the x-z plane of the frame U. The raw channel data frames acquired with the probe were processed to obtain an estimate U\hat{p}(n) of the position of the needle tip in the ultrasound probe frame U and a confidence measure d(n)∈(0, 1) of the estimate. We refer to this confidence measure as the validity of the estimate.

Process A used the amplitude-based approach previously developed to estimate the needle tip position and assess its validity. A photoacoustic image was recreated from the acquired channel data using delay-and-sum beamforming. The beamformed image was normalized and a binary threshold of 0.7 was applied to the normalized image. Binary erosion and dilation were performed with a 3×3 kernel to remove single pixel regions and connect segments which became disconnected during the binary threshold application. The erosion and dilation filters helped to ensure that the segmented needle tip was displayed as a single large component, rather than as multiple smaller components. Connected components were then labeled and their corresponding pixel areas were computed. If only one region was larger than 3 times the average area, then that region was assumed to be the needle tip and the centroid of that region was output as the needle tip position. Otherwise, the needle tip was assumed to be outside the field of view of the probe. For robustness, the estimated needle tip position was compared across five consecutive frames. If the needle tip was visible in each frame (i.e., d(n)=1) and the estimated position of the needle tip did not change by more than 1 cm across the 5 frames, then the needle tip position was labeled as valid.

Process B used a convolutional neural network (CNN) to provide estimates of the needle tip position and corresponding confidence levels in the range 0 to 1. With a focus on proving the feasibility of integrating deep learning-based approaches with real-time photoacoustic visual servoing systems, we used the ResNet-101 architecture and the Faster-RCNN detection method, which were previously demonstrated as an offline technique applied to photoacoustic channel data obtained with an E-CUBE 12R ultrasound scanner (Alpinion Medical Systems, Seoul, South Korea). For robustness, the estimated needle tip was compared across 5 consecutive frames as described above. If the needle tip was visible with a confidence level d(n)>0.7 in each frame and the estimated position of the needle tip did not change by more than 1 cm across the 5 frames, then the needle tip position was labeled as valid.

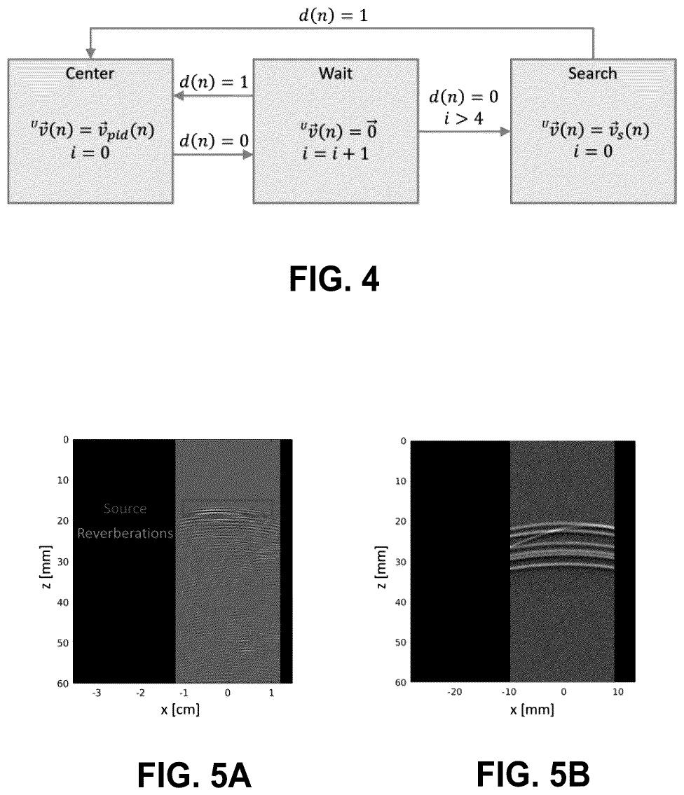

FIG. 4 shows the finite state machine used to control the translational degrees of freedom corresponding to the lateral and elevation dimensions of the probe. Two-dimensional (2D) photoacoustic images do not contain elevation displacement information. As a result, both Process A and Process B output zeros in the y-dimension of the estimate U \hat{p}(n). In the nominal “Center” state of the FSM, the error Uₑ₍ₙ₎ in the frame U was computed using the equation:

$${}^{U} \vec{e} ( n ) = [ \begin{matrix}1 & 0 & 0 \\ 0 & 0 & 0 \\ 0 & 0 & 0\end{matrix} ] ( {}^{U} \hat{p} ( n ) - ( {}^{U} {\vec{p}}_{cmd} ) , \qquad ( 1 )$$

where Uₚ_{_{cmd}}₌₀ is the desired position of the needle tip in the probe frame U. This computation of Uₑ₍ₙ₎ ensures that the visual servoing system will center the probe laterally above the needle tip without changing the axial or elevation displacement between the probe and the needle tip. If the FSM was in the “Center” state and the needle tip position estimate was marked as valid (i.e., d(n)=1 for Process A and d(n)>0.7 for Process B), then the end effector of the robot was commanded to move along the x-axis of the probe frame U with the velocity v_{pid}(n) given by the equation

$${\vec{v}}_{pid} ( n ) = {K}_{p} {}^{U} \vec{e} ( n ) + {K}_{i} {\sum }_{k = 0}^{n} {}^{U} \vec{e} ( k ) Δ T + {K}_{d} ( \frac{{}^{U} \vec{e} ( n ) - {}^{U} \vec{e} ( n - 1 )}{Δ T} ) , \qquad ( 2 )$$

where Kₚ, Kᵢ, and K_{d} are the gains of the PID controller (with values of 0.1, 0.01, and 0.001, respectively), and ΔT is the sampling time of the PID controller. The controller was executed every 0.1 s to match the pulse repetition rate of the laser.

The validity (i.e., d(n)) was used to indicate movement of the needle tip outside of the imaging plane of the probe. If the estimated needle tip position was marked as invalid, the FSM entered the “Wait” state. In this state, the end effector was held stationary until up to 5 frames of channel data were acquired by the photoacoustic imaging system. If a valid estimate of the needle tip position was obtained during that time, the FSM returned to the “Center” state. Otherwise, the FSM entered the “Search” state. In this state, the robot end effector was moved in a 2D spiral pattern given by

$${\vec{v}}_{s} ( n ) = [ \begin{matrix}An cos ( ω n ) \\ An sin ( ω n ) \\ 0\end{matrix} ] \qquad ( 4 )$$

where A and ω are the parameters of the spiral search pattern.

The commanded velocity Uᵥ(n) was then converted to the base frame B of the robot using the equation

$${}^{B} \vec{v} ( n ) = {}^{B} {T}_{E} ( n ) {}^{E} {T}_{U} {}^{U} \vec{v} ( n ) \qquad ( 4 )$$

where E_{T}_{_{U}} is the transformation from the ultrasound probe frame U to the robot end effector frame E and B_{T}_{_{E}}₍ₙ₎ is the instantaneous transformation from the frame E to the robot base frame B. The commanded velocity Bᵥ₍ₙ₎ was then transmitted to the internal velocity controller of the robot over the ROS topic for velocity commands to which the controller subscribed.

TABLE I
RANGE AND INCREMENT SIZES
OF SIMULATION VARIABLES

| Parameter | Min | Max | Increment |
|---|---|---|---|
| Depth Position (mm) | 5 | 55 | 0.3 |
| Lateral Position (mm) | −18.9 | 18.9 | 0.189 |
| Number of Reverberation Artifacts | 3 | 5 | 1 |
| Depth of Reverberation Artifacts | 1 | 10 | 0.3 |
| under Source (mm) | | | |
| Speed of Sound (m/s) | 1440 | 1640 | 6 |
| Object Intensity (Multiplier) | 0.75 | 1.1 | random |
| Channel SNR (dB) | −5 | 2 | random |

### B. Training the Convolutional Neural Network

Simulations that mimic the physics of photoacoustic wave propagation offer the ability to generate training data without the time-intensive process of experimentally gathering and hand-labeling the large datasets. This ease of data generation makes simulations a powerful tool in the context of deep learning. To train the CNN for Process B, 20,000 frames of photoacoustic channel data were generated using the k-Wave toolbox in MATLAB. We simulated a single source of diameter 0.1 mm and 4-6 artifacts in each image. One of the artifacts could be anywhere in the image to simulate a reflection artifact and maintain consistency with previous implementations. The remaining artifacts were constrained to the range 1 mm to 10 mm below the source to simulate the reverberation artifacts, as observed in FIG. 5(a), which shows one of the acquired channel data frames used as a reference to generate our training dataset. The ranges and increment values of our simulation variables are listed in Table I. We simulated a discrete ultrasound probe model with a sampling frequency of 11.88 MHz, an aperture of 128 elements, an element width of 0.25 mm, and an inter-element spacing of 0.05 mm. These parameters were selected to match the specifications of the Verasonics P4-2v probe to improve network performance. An example of our simulated training data is shown in FIG. 5(b).

The Detectron platform was utilized for training and validation. The network was initialized with pre-trained ImageNet weights and trained on 80% of the simulated images. The remaining 20% of the images were used for network validation. Finally, the Detectron-ROS package was utilized to incorporate the trained network into Process B of the visual servoing system.

### III. Experimental Methods

### A. Probe Centering Experiment

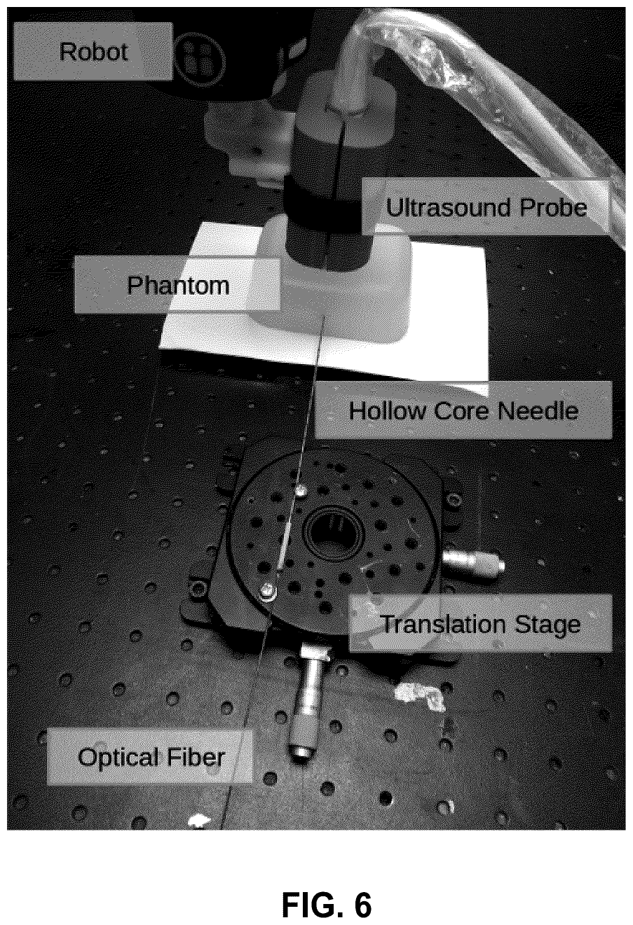

The experimental setup for the probe centering experiments is shown in FIG. 6. These experiments were implemented to estimate the probe centering and needle tracking errors of the two processes (i.e., A and B) for needle tip detection, similar to previous experiments implemented with a segmentation-based photoacoustic visual servoing system. The choices for each experimental trial included laser fluence (18.4 μJ/cm² or 49.5 μJ/cm²), needle tip detection process (Process A or B), and imaging environment (plastisol phantom or ex vivo chicken breast). There were 9 probe centering trials per fluence, per process, per imaging environment.

At the start of each experimental trial, the translation stage was reset to 0 mm. The fiber-needle pair was inserted into the chosen imaging environment. The ultrasound probe was placed on the surface of the imaging environment, with the imaging plane of the probe placed to contain as much of the intended trajectory of the needle tip as possible. The probe was then manually displaced distances of 2-10 mm from the needle tip in 2 mm increments in the lateral probe dimension, followed by initiation of visual servoing with Process A or B.

The visual servoing system was executed to center the probe above the needle tip. If the needle tip detection process output 5 consecutive valid estimates of the needle tip position (i.e., d(n)=1 for Process A and d(n)>0.7 for Process B), the trial was marked as a success. The mean of the lateral components of those 5 valid estimates U_{\hat{p}(n)} was computed, and the magnitude of U_{\hat{p}(n)} was output as the probe centering error. If 5 consecutive valid readings could not be obtained (i.e., d(n)=0 for Process A and d(n)<0.7 for Process B), the trial was marked as a failure. The mean and standard deviation of the probe centering errors were computed for each process and imaging environment.

### B. Needle Tip Tracking Experiment

The same setup shown in FIG. 6 and described in Section III-A was used for the needle tip tracking centering experiments, using the same choices for each experimental trial. There were similarly 9 needle tracking trials per fluence, per process, per imaging environment. After successfully centering the probe on the needle tip (as defined in Section III-A), the translation stage was used to move the needle tip in 2 mm increments along the lateral dimension of the probe. At each position, the output of the needle tip detection process was observed. If the process output five consecutive valid estimates of the needle tip position (i.e., d(n)=1 for Process A, and d(n)>0.7 for Process B), then the position was marked as a success. The needle tracking error was then computed using the equation:

$$e = ‖ {}^{B} {\vec{p}}_{f} - {}^{B} {\vec{p}}_{i} - {}^{B} {\vec{s}}_{n} ‖ , \qquad ( 5 )$$

where θ, Bₚᵢ, B_{f}, and Bₛ_{ₙ} are the needle tracking error, the initial robot end effector position, the final robot end effector position, and the measured displacement of the needle tip, respectively, in the robot base frame B.

If five consecutive valid readings could not be obtained (i.e., d(n)=0 for Process A and d(n)≤0.7 for Process B), the position was marked as a failure. The failure rates of Processes A and B were compared to assess the robustness of each algorithm.

### IV. Results

Table II summarizes the mean and standard deviation of probe centering errors for 90 trials per Process A or B, implemented with either the plastisol phantom or the ex vivo tissue. For each imaging environment, the probe centering errors of Processes A and B were within 0.1 mm of each other. The probe centering errors were similarly within 0.1 mm across the two imaging environments.

TABLE II
PROBE CENTERING ERRORS

| Process | Test Case | Mean Error [mm] | Std Dev [mm] |
|---|---|---|---|
| A | Phantom | 0.11 | 0.12 |
| A | Ex Vivo Tissue | 0.16 | 0.14 |
| B | Phantom | 0.19 | 0.16 |
| B | Ex Vivo Tissue | 0.18 | 0.17 |

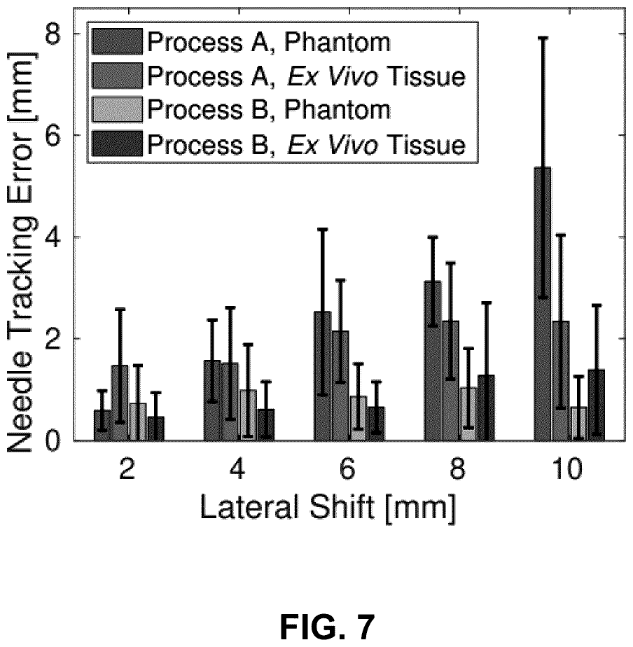

FIG. 7 shows the mean and standard deviation of the needle tracking errors for 18 trials per process, imaging environment, and lateral shift value. Process A produced needle tracking errors ranging 0.59-5.36 mm with a mean of 2.63 mm across all phantom trials and ranging 1.47-2.34 mm with mean of 1.96 mm across all ex vivo tissue trials. Process B generally produced better needle tracking errors than that of Process A, ranging 0.65-1.03 mm with a mean of 0.85 mm across all phantom trials and ranging 0.46-1.39 mm with a mean of 0.88 mm across all ex vivo tissue trials.

Table Ill lists the failure rates during needle tip tracking. For multiple trials with Process A, the needle tip was incorrectly labeled a reflection artifact that formed a larger bright region than the needle tip. This mislabeling caused a majority of the observed failures of Process A in both the phantom and the ex vivo tissue. Process B generally produced lower failure rates than Process A, with a mean improvement of 60.6% across all lateral shifts and both imaging environments. The highest failure rates of 1.85% and 3.70% for Process B were observed at a lateral offset of 10 mm in the phantom and ex vivo tissue environments, respectively. These failure locations are consistent with previous reports demonstrating increased CNN failure rates as lateral offset from the center of an image increases, due to a reduced number of source examples on the image periphery.

TABLE III
NEEDLE TRACKING FAILURE RATES

| | | |
|---|---|---|
| Lateral | Process A | Process B |

| Shift [mm] | Phantom | Ex Vivo | Phantom | Ex Vivo |
|---|---|---|---|---|
| 2 | 3.51% | 0.00% | 0.00% | 0.00% |
| 4 | 7.02% | 0.00% | 0.00% | 1.85% |
| 6 | 7.02% | 0.00% | 0.00% | 0.00% |
| 8 | 5.26% | 1.85% | 0.00% | 1.85% |
| 10 | 3.51% | 1.85% | 1.85% | 3.70% |

We additionally note the <100 ms execution time requirement for Process B in order to achieve the same 10 Hz frame rate previously demonstrated with visual servoing systems using iterations of Process A. This requirement is dictated by the 10 Hz laser pulse repetition frequency of the photoacoustic imaging system. The mean±one standard deviation of execution times from 36 trials of both experiments described above with Process B were 75.2±12.8 ms and 73.9±13.2 ms per channel data frame with the phantom and ex vivo tissue, respectively.

### V. Discussion

The results presented in this manuscript highlight the potential of a CNN to provide an alternative input to command robotic visual servoing systems. This potential was demonstrated with a system composed of a Verasonics ultrasound engine, which was not used in any previous work testing similar CNN architectures. It is promising that the presented needle tracking errors are comparable to the 0.40±0.22 mm point source location errors obtained by others with an Alpinion E-CUBE 12R ultrasound scanner and an L3-8 probe. This similar success indicates that the previously proposed deep learning methods for photoacoustic point source detection are generalizable across multiple imaging system platforms.

We identified three advantages of integrating this novel deep learning approach with a robotic photoacoustic visual servoing system, when compared to the amplitude-based image segmentation approach: (1) lower tool tip tracking failure rates in the presence of reflection artifacts, (2) reduced needle tracking errors across different imaging environments, and (3) maintenance of 10 Hz frame rates despite increased algorithmic complexity. Regarding the first advantage, the sensitivity of the beamforming techniques to reflection artifacts (e.g., caused by bone) can lead to uncertain and potentially hazardous robot arm movements during surgical procedures, which is a major concern for the steps required to complete the segmentation-based visual servoing approach (i.e., Process A). Instead of adding successive layers of complexity to the beamformer or segmentation algorithm to account for these artifacts and features, the deep learning approach (i.e., Process B) trains a CNN to distinguish between true sources and reflection artifacts in the raw channel data, thus mitigating the introduction of misclassification errors.

To appreciate the second advantage, the reduced mean needle tracking errors with the deep learning approach (i.e., 0.85 mm and 0.88 mm in phantom and ex vivo tissue, respectively) can be compared to the needle tracking errors obtained with the segmentation approach (i.e., 2.63 mm and 1.96 mm in the phantom and ex vivo tissue, respectively). The overall mean improvement with Process B translates to 67.7% and 55.3% reductions in needle tracking errors in the phantom and ex vivo tissue, respectively.

With regard to the third advantage of achieving 10 Hz frame rates, the deep learning approach has a higher algorithmic complexity compared to the image segmentation approach (i.e., O(MNS) for Process A vs. O(Nconv D² N²^{_{ch}} MN) for Process B, where M, N, S, and Nconv are the numbers of receiving elements, samples acquired per frame, scan lines in the beamformed image, and convolutional layers in the CNN, respectively, D is the size of the largest convolutional kernel, and N_{ch} is the maximum filter dimension). This constraint would ordinarily compromise the maximum achievable frame rate. However, the use of GPUs combined with recent deep learning advances allow us to benefit from the deep learning approach without compromising the desired frame rate dictated by the 10 Hz laser pulse repetition frequency.

Future possible improvements to the proposed deep learning visual servoing system include mitigating tracking errors obtained with larger lateral displacements from the center of the probe and increasing the number of degrees of freedom for the motion of the robot end effector. Regarding tracking error mitigation, an increase in the lateral displacement of the source from the center of the probe during the ex vivo experiments resulted in needle tracking errors increasing from 0.46 mm to 1.39 mm (FIG. 7) and needle tracking failure rates increasing to a maximum of 3.70% (Table III). This increase in error may potentially be resolved by improving the training process and by increasing the number of training images containing sources with large lateral offsets. Regarding the tracking degree of freedom, the nominal motion of the robot end effector is limited to 1 dimension in our visual servoing system, and a second dimension is used to search for and find the tool tip when it is not in the imaging plane of the probe. While these two degrees of freedom sufficiently achieve the desired end result, future work will determine the extent to which additional degrees of freedom are necessary to achieve more complicated path planning outcomes with the proposed deep learning-based photoacoustic visual servoing system.

### VI. Conclusion

This example is the first to demonstrate the integration of deep learning-based techniques with photoacoustic-based robotic visual servoing of needle tips. The deep learning-based needle tip detection process is more accurate (e.g., 0.46-1.39 mm needle tracking errors) and produces lower failure rates (e.g., 0-3.70%) when compared to the alternative photoacoustic image segmentation-based visual servoing system (which produced tracking errors and failure rates of 0.59-5.36 mm and 0-7.02%, respectively). The deep learning-based system additionally maintains the frame rates achieved with the segmentation-based approach. Overall, these results demonstrate the promise of a robotic photoacoustic visual servoing system that bypasses traditional image formation and segmentation steps, instead supplying robot controller input based on details contained within raw photoacoustic sensor data. While this work focuses on tracking needle tips, the system described herein can be extended to track the tips of catheters and a multitude of other surgical tools that are critical to automated surgeries and interventional procedures.

### Example 2: Theoretical Framework to Predict Generalized Contrast-to-Noise Ratios of Photoacoustic Images with Applications to Computer Vision

### I. Introduction

The integration of computer vision with medical imaging is a critical subfield of modern healthcare. Applications of computer vision algorithms in healthcare include classifying disease in medical images, segmenting image features of interest to alert surgeons during an operation, and tracking targets of interest in robot-assisted procedures, which has the potential to reduce infection rates, shorten hospital stays, and improve postoperative function. These advantages are applicable across multiple medical imaging modalities (e.g., magnetic resonance, X-ray, ultra-sound, and photoacoustic imaging), and performance characterization is important for overall system design.

Amplitude-based segmentation of a target from the background of an image is an example computer vision-based task with widespread applications. Others have used this technique to track needle tips with an ultrasound-based robotic visual servoing system. Ultrasound has numerous advantages over other imaging modalities, including its low cost, high frame rates, and the lack of ionizing radiation associated with other modalities, such as fluoroscopy. However, ultrasound imaging fails in acoustically challenging environments characterized by significant acoustic clutter, sound scattering, and sound attenuation. Under these conditions, the performance of computer vision algorithms (e.g., target segmentation) inherently suffers.

Photoacoustic imaging is a more recent imaging modality that combines optical transmission with ultrasound reception to overcome the traditional ultrasound imaging limitations noted above. The photoacoustic imaging process is initiated when a light source (typically a nanosecond-pulsed laser) illuminates a region of interest. The illuminated tissue absorbs the light, undergoes thermal expansion, and produces a pressure gradient that is received by an ultrasound transducer, then reconstructed into an image. Recent advances in photoacoustic imaging demonstrate promise to mitigate the risks associated with complex surgical procedures, including liver resections, spinal fusion surgeries, and gynecological surgeries. Previous work from our group also demonstrates successful tracking of needle tips and catheter tips using a photoacoustic-based robotic visual servoing system. The performance of our visual servoing system directly depends on the performance of the target segmentation algorithm, which, in turn, depends on the detectability of targets in the acquired photoacoustic images. The detectability of targets in photoacoustic images has traditionally been quantified using image quality metrics, such as signal-to-noise ratio (SNR), contrast, and contrast-to-noise ratio (CNR). However, these metrics are not bounded, are sensitive to common image manipulation techniques (e.g., dynamic range adjustment and thresholding), and have substantial variability when comparing reported numbers to the detectability of photoacoustic image targets.

Others developed the generalized contrast-to-noise ratio (gCNR) to assess the probability of lesion detection in ultrasound images and presented a theoretical framework to predict the gCNR of ultrasound images. Subsequent contributions from our group demonstrated the applicability of gCNR to photoacoustic images created with delay-and-sum (DAS), short-lag spatial coherence (SLSC), generalized coherence factor weighting applied to DAS, and minimum variance beam-formers. However, these contributions used histogram-based approximations of the ground truth. These approximations, which we refer to as measured gCNR, are sensitive to the selection of bin widths. To overcome this limitation, we require a theoretical framework to predict the true gCNR in photoacoustic images. The ultrasound gCNR theory is insufficient for photoacoustic images for two primary reasons. First, photoacoustic target characteristics differ from those of ultrasound targets. The exponential probability distribution in does not sufficiently model the target power distribution in photoacoustic images. Second, the theoretical ultrasound framework is not equipped to solve the multiple decision boundaries that exist when classifying photoacoustic images.

In particular, the gCNR metric depends on target and background distributions, which depend on photoacoustic system parameters such as laser energy, receiver characteristics, and image processing algorithms implemented to improve image quality. Our previous conference papers individually demonstrated a subset of these dependencies, including the relationships among gCNR, laser energy, and frame averaging, relationships between gCNR and channel SNR, and relationships between image quality metrics (i.e., gCNR, CNR, contrast, and SNR) and target segmentation. Considering that gCNR was developed to assess the probability of detecting targets of interest and to overcome limitations of traditional image quality metrics, we hypothesize that gCNR has superior potential to predict the performance of a photoacoustic-based visual servoing system in comparison to more traditional alternatives.

To investigate this hypothesis, we first developed a general framework to relate system parameters (e.g., laser energy, channel SNR, and target and background power distributions) to computer vision-based task performance. The three additional contributions that follow from this general framework include: 1) a theoretical derivation to predict the gCNR of photoacoustic images using models of the target and background power distributions; 2) characterization of the connections among system parameter settings, image characteristics, and the computer vision goal of performing automated target segmentation; and 3) implementation of theory and characterizations to predict target segmentation performance from experimental photoacoustic images.

The remainder of this example is organized as follows: Section II presents our general framework to describe the relationships among the components of a photoacoustic visual servoing system, followed by our theoretical derivation of gCNR predictions in photoacoustic images and our theoretical description of the thresholding step typically implemented prior to initiating a computer vision task. Section III details the simulation of photoacoustic targets and acquisition of experimental and in vivo data, followed by methods to implement the theoretical gCNR predictions and validate these predictions with experimental measurements. Section IV details the results of the presented methods. Section V discusses the implications of this work and its future potential. Finally, Section VI concludes this article with a summary of our findings.

### II. Theory

### A. General Framework

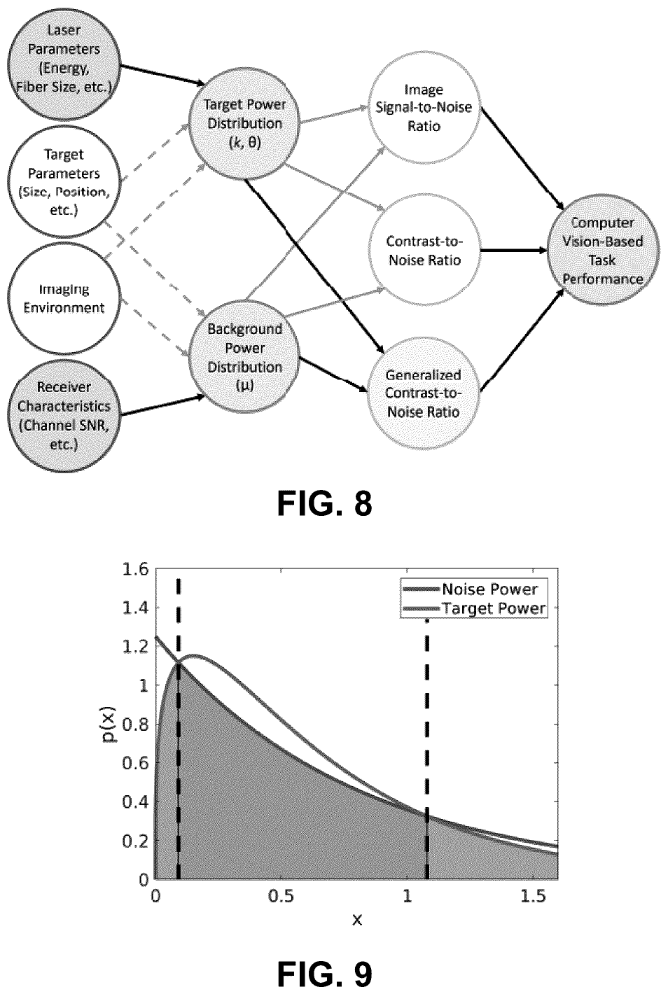

The major components of a photoacoustic-based computer vision system can be represented as a directed graph highlighting multiple relationships among system properties, as illustrated in FIG. 8. There are four color-coded layers in the graph representing different properties of a photoacoustic-based computer vision system. The first layer represents baseline system parameters and imaging environment details, such as receiver characteristics, laser parameters, target dimensions, and target depth. The second layer corresponds to the signal characteristics observed in the acquired photoacoustic image. The third layer represents possible image quality metrics for photoacoustic images. The fourth layer represents the performance of a computer vision-based task (e.g., target segmentation) on the given photoacoustic image.

The nodes in each layer of FIG. 8 describe properties that are directly impacted by one or more nodes in the preceding layer. These dependencies are indicated by the directed lines (i.e., edges) which connect the related pairs of nodes. The solid black lines denote relationships reported in this example, including: 1) the relationship between channel SNR and background power; 2) the relationship between laser energy and target power; 3) the relationships among target power, background power, and gCNR (with mathematical descriptions for these relationships reported in Section II-B); and 4) the relationship between image quality metrics (i.e., SNR, CNR, or gCNR) and photoacoustic target segmentation (with mathematical descriptions for the impact of thresholding prior to segmentation reported in Section II-C). The third and fourth relationships are foundational to predicting the performance of photoacoustic-based visual servoing of needle tips, catheter tips, and surgical tool tips. In addition to providing mathematical descriptions associated with these foundational relationships, all relationships indicated by the black lines in FIG. 8 will be demonstrated empirically in Section IV.

The gray lines in FIG. 8 denote connections that are expected to exist, with solid gray indicating known mathematical expressions that are not reported in this article and dashed gray indicating relationships which were empirically observed in this article, yet difficult to isolate and quantify due to multiple confounding effects on the resulting photoacoustic images.

B. Modeling gCNR Predictions as a Classification Task

1) Approach Overview: The gCNR metric has been introduced and described as a normalized measurement of the highest achievable probability of success of an optimal two-class classifier operating on a given image. The associated expression is

$$gCNR = 1 - 2 {P}_{e} \qquad ( 1 )$$

where Pe is the probability of error of the classifier. Deter-mining Pe requires characterization of this classifier, based on decision boundaries, which are computed from the points of intersection between the probability distribution functions (PDFs) of target and background powers. Considering that histogram-independent gCNR predictions depend on these power distribution models, an accurate gCNR prediction will demonstrate the accuracy of our chosen target and background power distribution models. Considering the additional dependencies on baseline system parameters and imaging environment details (located in the first layer of FIG. 8), accurate gCNR predictions will additionally establish the validity of our overall framework.

To predict the gCNR of a photoacoustic image, a four step approach is used: 1) modeling the target power distribution (Section II-B2); 2) modeling the background power distribution (Section II-B3); 3) determining decision boundaries computed from the points of intersection between the target and background power distributions (Section II-B4); and 4) computing the gCNR predictions based on the decision boundaries (Section II-B5).

2) Target Power Distribution: Others demonstrated that as a consequence of photoacoustic speckle, the envelope of photoacoustic radio frequency (RF) signals can be modeled by the Nakagami-m distribution. In this model, the signal power then follows the gamma distribution, which is a generalization of the exponential distribution employed to characterize ultrasound image targets in [24]. The PDF of the photoacoustic target power distribution, pi, can be written as

$${p}_{i} ( x ; k , θ ) = \frac{{x}^{k - 1} {e}^{- \frac{x}{θ}}}{{θ}^{k} Γ ( k )} \qquad ( 2 )$$

where x is the target power, and k and θ are scalar parameters denoting the shape and scale of the gamma distribution, respectively. The mean of the target power distribution, μ^{x}, is

$${μ}^{′} = k θ . \qquad ( 3 )$$

Note that when k=1, the gamma distribution reduces to an exponential distribution with μ^{x}=θ.

3) Background Power Distribution: We assume that the back-ground of a photoacoustic image primarily contains thermal and electronic noise. Others demonstrated that thermal noise can be modeled using the complex normal distribution in ultrasound images created using DAS beam-forming. Given the similar receiver hardware and software, it is reasonable to expect the thermal noise in DAS photoacoustic images to also follow the complex normal distribution. Therefore, the combined sum of the thermal and electronic noise can be modeled using a complex normal distribution. The real and imaginary (i.e., in-phase and quadrature, or IQ) components of the image noise are assumed to be independent and identically distributed with mean zero and variance σ². In this case, the amplitude of the background signal follows the Rayleigh distribution, and the background power can be modeled using the exponential distribution, which yields the following PDF:

$${p}_{o} ( x ; μ ) = \frac{1}{μ} {e}^{- \frac{x}{μ}} \qquad ( 4 )$$

where pₒ is the PDF of the background power distribution, x is the background power, and μ is the mean noise power given by the following expression:

$$μ = 2 {σ}^{2} . \qquad ( 5 )$$

As an aside, to mathematically relate this background power distribution to the receiver characteristic of channel SNR (see FIG. 8), particularly when the photoacoustic imaging system acquires raw RF channel data, the associated channel noise is assumed to be Gaussian distributed with a mean of zero and a variance of σ²^{_{ch}}. The DAS beamforming process converts the channel data to an image by delaying the channel data and summing the delayed channel data across the received elements. Therefore, the following linear relationship between σ² and σ²^{_{ch}} is expected:

$${σ}^{2} = ( \frac{{N}^{2}}{2} ) {σ}_{ch}^{2} \qquad ( 6 )$$

where N is the number of receive elements, and the factor of 2 is introduced because of the conversion from RF to IQ data. Substituting (5) into (6) yields

$$μ = {N}^{2} {σ}_{ch}^{2} . \qquad ( 7 )$$

4) Computing Decision Boundaries: The PDFs of the target and background power of a DAS photoacoustic image are rep-resented by the orange and blue lines, respectively, in FIG. 9. The parameters corresponding to these target and background power distributions are k=1.3, θ=0.5, and μ=0.8. The two points of intersection between the PDFs are highlighted by the black dashed lines in FIG. 9. The abscissae of these points of intersection form the set of decision boundaries, D, of an ideal two-class classifier for the sample image, defined as

$$D = \{ {ϵ}_{m} : m = 0 , 1 \} \qquad ( 8 )$$

where Eₘ denotes the abscissae of the points of intersection.

The solutions for Eₘ can be computed by equating the target power distribution, pᵢ, to the background power distribution, pₒ, as follows:

$${p}_{i} ( {ϵ}_{m} ; k , θ ) = {p}_{o} ( {ϵ}_{m} ; μ ) . \qquad ( 9 )$$

Substituting the expressions for the target and background probability distributions from (2) and (4) into (9) yields the following expression for the decision boundaries:

$${e}^{( \frac{1}{θ} - \frac{1}{μ} ) {ϵ}_{m}} = ( \frac{μ}{{θ}^{k} Γ ( k )} ) {ϵ}_{m}^{k - 1} . \qquad ( 10 )$$

Equation (10) can be satisfied with four possible cases of the decision boundary points, depending on the values of k, θ, and μ.

Case 1 (k|=1 and θ|=μ): In this first case, there are up to two decision boundaries, as shown in FIG. 9. To compute these decision boundaries, (10) may be simplified as

$${e}^{a {ϵ}_{m}} = b {ϵ}_{m}^{c} \qquad ( 11 )$$

where a=(1/θ)−(1/μ), b=μ/(θ^{k} f(k)), and c=k−1.

For nonzero values of a (i.e., θ|=μ), (11) is satisfied by the LambertW function as

$${ϵ}_{m} = - \frac{c}{a} \times Lambert W [ ( - \frac{a}{c} ) {( \frac{1}{b} )}^{\frac{1}{c}} ] . \qquad ( 12 )$$

Considering that (12) does not yield a simple closed-form expression for Eₘ, numerical values for Eₘ may be computed using the lambertw function in MATLAB. The complex outputs of this function can then be discarded to obtain up to two decision boundaries for the ideal two-class classifier when k|=1 and θ|=μ.

Case 2 (k|=1 and θ=μ): In this second case, (10) can be simplified to the following expression:

$${ϵ}_{m}^{k - 1} = {θ}^{k - 1} Γ ( k ) . \qquad ( 13 )$$

Because Eₘ is a decision boundary corresponding to a power value sampled from the image, we are interested in nonnegative, real-valued solutions to (13). The parameters of the target power distribution have the constraints k≥0 and θ≥0. From the constraint on the target shape parameter k, we obtain the related constraint f(k)≥0. With these constraints, (13) can be rearranged to obtain the following expression for the single-decision boundary E₀ when k|=1 and θ=μ:

$${ϵ}_{0} = {θ [ Γ ( k ) ]}^{\frac{1}{k - 1}} . \qquad ( 14 )$$

Case 3 (k=1 and θ|=μ): This third case is observed in images with low channel SNR. A decrease in the channel SNR of a photoacoustic image results in the background noise overwriting the target region, causing a similarity that allows both the target and background power distributions to be modeled using the exponential distribution. Therefore, the PDF described by (2) reduces to

$${p}_{i} ( x ; 1 , θ ) = \frac{1}{θ} {e}^{- \frac{x}{θ}} \qquad ( 15 )$$

which is the same as the target model for ultrasound images. Substituting (15) and (4) into (9) yields the following closed-form expression for the single-decision boundary E₀ when k=1 and θ|=μ:

$${ϵ}_{0} = μθ ( \frac{ln μ - ln θ}{μ - θ} ) . \qquad ( 16 )$$

Case 4 (k=1 and θ=μ): The fourth case occurs in photoacoustic images with sufficiently low channel SNR such that the target and background regions are indistinguishable. Similar to Case 3, the target power distribution is modeled as an exponential distribution with mean e. In this trivial case, the target and background power distributions coincide completely, resulting in infinitely many points of intersection between the distributions. As a result, no selection of decision boundaries can distinguish between the target and background signals under the conditions k=1 and θ=μ.

5) Predicting gCNR Using Decision Boundaries: Predictions of gCNR can be computed for the four cases described above. In each case, we assume that the target and background regions have equivalent areas.

For Case 1, Pₑ in (1) expands to

$${P}_{e} ( {ϵ}_{0} , {ϵ}_{1} ) = \frac{{P}_{F} ( {ϵ}_{0} , {ϵ}_{1} ) + {P}_{M} ( {ϵ}_{0} , {ϵ}_{1} )}{2} \qquad ( 17 )$$

where Pₑ(E₀, E₁) is the probability of error, and P_{F}(E₀, E₁) and P_{M}(E₀, E₁) are the probabilities of false positives and missed detections, respectively, for the given pair of decision boundaries (E₀, E₁). The probabilities P_{F}(E₀, E₁) and P_{M}(E₀, E₁) are represented by the blue and orange shaded regions, respectively, in FIG. 9. Expressions for these probabilities are derived based on the decisions of the optimal two-class classifier in the intervals [0, E₀], (E₀, E₁), and [E₁, ∞). In the interval (E₀, E₁), the optimal decision is to assign the target class to samples. As a result, the probability of false positives P_{F}(E₀, E₁) is

$${P}_{F} ( {ϵ}_{0} , {ϵ}_{1} ) = {\int }_{{ϵ}_{0}}^{{ϵ}_{1}} {p}_{0} ( x ; μ ) dx . \qquad ( 18 )$$

In the intervals [0, E₀] and [E₁, ∞), the optimal decision is to assign the background class to samples. Therefore, the probability of missed detections P_{M}(E₀, E₁) is

$${P}_{M} ( {ϵ}_{0} , {ϵ}_{1} ) = {\int }_{0}^{{ϵ}_{0}} {p}_{i} ( x ; k , θ ) dx + {\int }_{{ϵ}_{1}}^{∞} {p}_{i} ( x ; k , θ ) dx . \qquad ( 19 )$$

Substituting (18) and (19) into (17) yields

$${P}_{e} ( {ϵ}_{0} , {ϵ}_{1} ) = \frac{1}{2} ( \begin{matrix}{\int }_{0}^{{ϵ}_{0}} {p}_{i} ( x ; k , θ ) dx \\ + {\int }_{{ϵ}_{0}}^{{ϵ}_{1}} {p}_{o} ( x ; μ ) dx \\ + {\int }_{{ϵ}_{1}}^{∞} {p}_{i} ( x ; k , θ ) dx\end{matrix} ) . \qquad ( 20 )$$

The resulting expression for the gCNR of a photoacoustic target (after substitution into (1)) is

$$\begin{matrix}{gCNR}_{PA 1} = 1 - {\int }_{0}^{{ϵ}_{0}} {p}_{i} ( x ; k , θ ) dx \\ - {\int }_{{ϵ}_{0}}^{{ϵ}_{1}} {p}_{o} ( x ; μ ) dx \\ - {\int }_{{ϵ}_{1}}^{∞} {p}_{i} ( x ; k , θ ) dx .\end{matrix} \qquad ( 21 )$$

For Case 2, with the assumption that photoacoustic targets have a higher amplitude than the background, we classify samples in the intervals [0, _0) and [_0, ∞) as belonging to the background and target, respectively. Therefore, the probabilities of error are

$${P}_{F} ( {ϵ}_{0} ) = {\int }_{{ϵ}_{1}}^{∞} {p}_{o} ( x ; μ ) dx \qquad ( 22 )$$

$${P}_{M} ( {ϵ}_{0} ) = {\int }_{0}^{{ϵ}_{0}} {p}_{i} ( x ; k , θ ) dx \qquad ( 23 )$$

$${P}_{e} ( {ϵ}_{0} ) = \frac{{P}_{F} ( {ϵ}_{0} ) + {P}_{M} ( {ϵ}_{0} )}{2} \qquad ( 24 )$$

and the resulting expression for the gCNR of a photoacoustic target is

$${gCNR}_{PA 2} = 1 - {\int }_{0}^{{ϵ}_{0}} {p}_{i} ( x ; k , θ ) dx - {\int }_{{ϵ}_{0}}^{∞} {p}_{o} ( x ; μ ) dx . \qquad ( 25 )$$

The precise value of the integral in (25) depends on the values of k, θ, and μ.

For Case 3, (4), (15), and (16) are substituted into (25) to obtain the following closed-form expression for gCNR:

$${gCNR}_{PA 3} = {( \frac{μ}{θ} )}^{- \frac{μ}{μ - θ}} - {( \frac{μ}{θ} )}^{- \frac{θ}{μ - θ}} . \qquad ( 26 )$$

Comparing (4) and (15) with (28) and (27) in [24], we observe that photoacoustic images with low channel SNR values have statistical properties similar to ultrasound images. As a result, the final expression for gCNR obtained in (26) has a similar format to (35) in [A. Rodriguez-Molares et al., “The generalized contrast-to-noise ratio: A formal definition for lesion detectability,” IEEE Trans. Ultrason., Ferroelectr., Freq. Control, vol. 67, no. 4, pp. 745-759, April 2020], which was derived for ultrasound images of hypoechoic targets. However, photoacoustic signals tend to be brighter than their surrounding environments, which more closely aligns with the hyperechoic ultrasound target case described in [A. Rodriguez-Molares et al., “The generalized contrast-to-noise ratio: A formal definition for lesion detectability,” IEEE Trans. Ultrason., Ferroelectr., Freq. Control, vol. 67, no. 4, pp. 745-759, April 2020.].

For Case 4, no optimal decision boundaries can be obtained for a two-class classifier, which achieves an accuracy of at most 50% on these images using any available (nonoptimal) decision boundary. Under these conditions, it is trivial to prove that the gCNR of the underlying photoacoustic image is zero.

C. Effect of Thresholding on gCNR

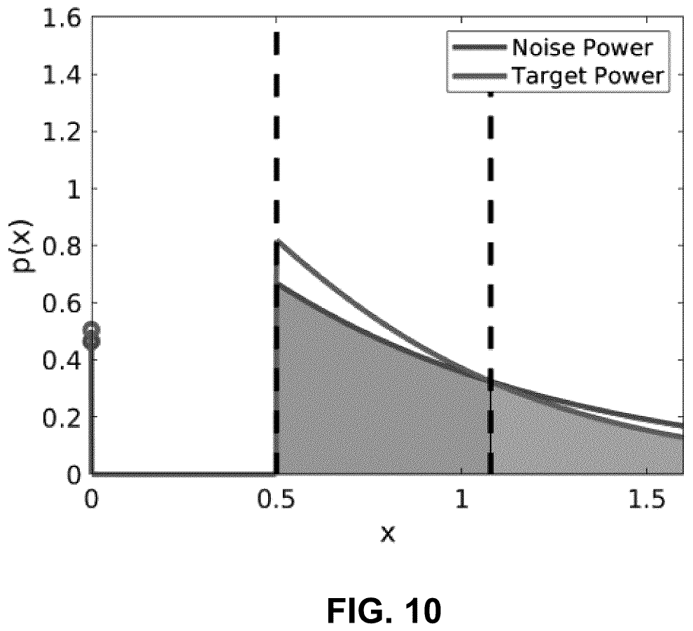

As reported elsewhere, applying a threshold of to t0 a photoacoustic image results in pixels with power values less than to in both the target and background regions of the image being set to zero. FIG. 10 shows the effect of the thresholding operation on the sample target and background distributions described in Section II-B4 and plotted in FIG. 9 with t₀=0.5. For target and background pixel power values x<t₀, the original target and background distributions in FIG. 9 are replaced by Dirac delta functions (t0 pᵢ(y)dy)δ(x)₀ and (t0 pₒ(y)dy)δ(x), respectively, in FIG. 10. The target and background power distributions remain unchanged for x>t0. The set of decision boundaries Dt of the optimal classifier for the thresholded image is formulated from to and D in (8) as follows:

$${D}_{t} = \{ {t}_{0} \} ⋃ \{ {ϵ}_{m} : {ϵ}_{m} ∈ D , {ϵ}_{m} > {t}_{0} \} . \qquad ( 27 )$$

As the threshold value to increases, the separability between the target and background power distributions is expected to decrease, resulting in an expected decrease in gCNR.

### III. Methods

### A. Validation Experiments

1) Datasets and Initial Imaging Environments: We acquired three datasets (i.e., simulated, experimental, and in vivo) to validate the theoretical derivations of gCNR predictions with gCNR measurements across a range of channel SNR values. The simulated dataset consisted of 6-mm-diameter photoacoustic targets residing at a depth of 15 mm using the k-Wave toolbox. Optical absorbers of size 6 μm were placed in the target region with a spatial density of 135 absorbers/mm². This absorber density lies between the moderately dense and dense cases of 3.18 and 318.31 absorbers/mm², respectively. A transducer was defined in the simulation with a 0.3-mm pitch, 0.06-mm kerf, 128 elements, a center frequency of 5 MHZ, and a bandwidth of 2-8 MHz. These parameters were chosen to match the Alpinion L3-8 transducer (Seoul, South Korea). Ten channel data frames were simulated with absorber positions varied within the target region.

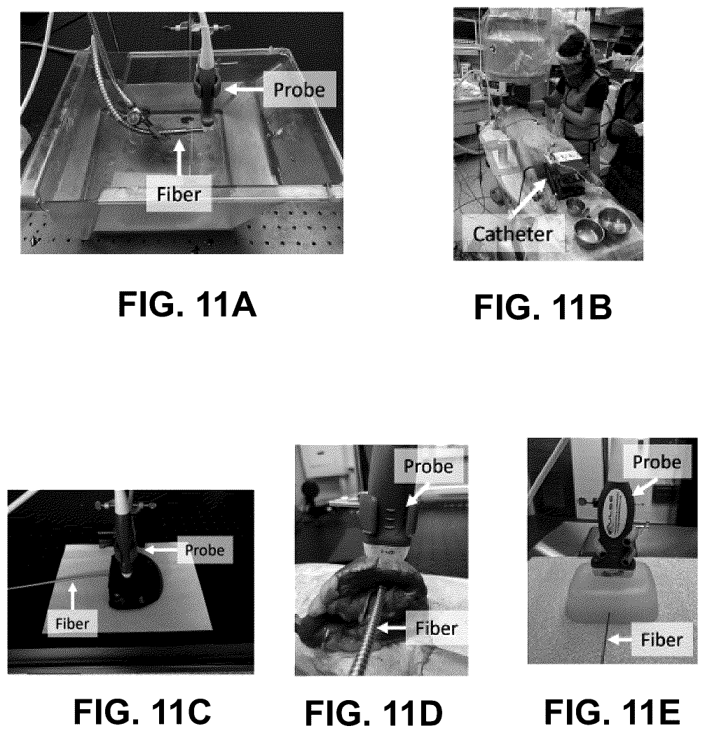

The experimental dataset was acquired using the setup shown in FIG. 11(a). The photoacoustic imaging system used for this dataset consisted of an Alpinion E-CUBE 12R ultrasound system connected to an Alpinion L3-8 linear array transducer, and a Phocus Mobile laser (OPOTEK, Carlsbad, CA, USA) connected to a 5-mm-diameter optical fiber bundle. The free end of the fiber bundle was submerged in a water bath. The laser was pulsed at a fixed repetition rate of 10 Hz, with a fixed wavelength of 760 nm. The laser energy at the fiber bundle tip was varied from 15 to 68 mJ, as measured with an Ophir PE50BF-DIFH-C energy meter (North Logan, UT, USA), which was used for all reported energy measurements unless otherwise stated. The laser output a trigger signal with each pulse to synchronize the acquisition of data by the ultrasound system. Ten frames of photoacoustic data were acquired with the tip of the fiber bundle at a depth of 35 mm in the imaging plane of the transducer.

The in vivo dataset was acquired during an in vivo cardiac catheterization procedure conducted on a Sus domesticus domestic swine weighing 78 lbs, as shown in FIG. 11(b). All in vivo procedures were approved by the Johns Hopkins Institutional Animal Care and Use Committee. The swine was anesthetized and subsequently intubated to provide gas anesthesia throughout the surgery. Respiration was controlled with a ventilator that supplied medical air. The blood pressure and heart rate of the swine were monitored throughout the surgery and imaging procedures. The photoacoustic imaging system (consisting of the Alpinion E-CUBE 12R ultrasound scanner and Opotek Phocus Mobile laser) was placed next to the operating table. The ultrasound system was connected to a 64-element Alpinion SP1-5 phased array transducer with a center frequency of 3 MHz and a sampling frequency of 40 MHz. One end of a 1-mm-diameter optical fiber was inserted into a 2.5-mm-diameter steerable cardiac catheter. The other end of the fiber was interfaced with the laser. An incision was created to gain access to the femoral vein. The catheter was then guided through the inferior vena cava toward the heart with the ventricular septum as the final destination. A fixed laser wavelength of 750 nm was chosen to visualize the catheter tip inside the heart. Photoacoustic data were acquired with laser energies at the fiber tip varied from 2.9 to 608.5 μJ. Data from this animal study were previously described.

Each of the simulated, experimental, and in vivo datasets consisted of ten frames of raw RF channel data. Gaussian-distributed noise was added to each channel data frame in these three datasets to obtain channel SNR values in the range −40 to 40 dB with increments of 1 dB. Ten noisy channel data frames were generated per channel SNR level for each of the simulated or acquired channel data frames, resulting in 8100 noisy channel data frames per dataset.

The k-Wave toolbox was utilized to add noise to raw RF channel data frames with standard deviation σ_{ch} defined using the following equation:

$${σ}_{ch} = \sqrt{\{ {s}_{ch}^{2} \}} \times {10}^{- \frac{{SNR}_{ch}}{20}} \qquad ( 28 )$$

where s_{ch} is the raw channel data signal prior to the addition of noise, and SNR_{ch} is the desired channel SNR value.

2) Laser Energy Variations: The relationship between gCNR and laser energy was investigated with two additional imaging environments (i.e., a plastisol phantom and an ex vivo caprine heart shown in FIG. 11(c) and(d), respectively). The photoacoustic imaging system used for these experiments consisted of an LS-series pulsed laser diode (PLD) (Laser Components, Bedford, NH, USA) and an Alpinion E-CUBE 12R ultrasound scanner connected to an Alpinion SP1-5 transducer. One end of a 2-mm-diameter fiber bundle was interfaced with the laser. The other end was inserted into each imaging environment. A function generator transmitted a trigger signal to both the ultrasound scanner and the PLD at a frequency of 20 Hz. Two 5-V channels from a regulated power supply unit (GW Instek, New Taipei City, Taiwan) were connected to the PLD to control the pulsewidth and peak power of the laser. The laser energy settings were varied to maximize the range of achievable gCNR in each environment, resulting in 99 unique pairs of pulsewidth and peak energy for the phantom environment and 88 unique pairs for the ex vivo environment. For each laser energy setting, 100 frames of photoacoustic data were acquired by the ultrasound system. The corresponding laser energies at the fiber bundle tip were then measured using a J-10MT-10 KHZ energy meter by Coherent, Inc., Santa Clara, CA, USA, resulting in ranges of 0.07-10.95 and 0.37-11.36 μJ for the phantom and ex vivo environments, respectively. Unless otherwise stated, this entire energy range is reported in our results for this experiment. The photoacoustic imaging system consisting of the PLD and Alpinion ultrasound scanner was previously used by others to demonstrate a photoacoustic-based, teleoperated, robotic surgical system.

3) Segmentation Accuracy Experiments: The relationships between the accuracy of the target segmentation process and selected image quality metrics (i.e., gCNR, SNR, and CNR) were investigated with the in vivo dataset described in Section III-A1 as well as with an alternative plastisol phantom shown in FIG. 11(e). The photoacoustic imaging system for this phantom experiment consisted of the Phocus Mobile Laser and Alpinion E-Cube 12R scanner connected to the Alpinion L3-8 transducer described in Section III-A1. A 1-mm-diameter optical fiber was inserted into the phantom. The other end of the optical fiber was interfaced with the laser. Photoacoustic channel data were acquired using a fixed wavelength of 750 nm, a pulse repetition rate of 10 Hz, and laser energies at the fiber tip ranging from 13.5 μJ to 2.3 mJ.

### B. Image Formation, Analysis, and Segmentation

For each dataset described in Section III-A, photoacoustic images were reconstructed with DAS beamforming, followed by envelope detection, normalization, log compression, and thresholding prior to image display. For each beamformed photoacoustic image, circular regions of interest (ROIs) were selected within the target and background using either a manual or automated approach. The manual ROI approach was implemented for experiments demonstrating relationships between nodes in the first, second, and third layers of FIG. 8. Automated ROI selection was used to demonstrate relationships between nodes in the third and fourth layers of FIG. 8.

To create manual ROIs, two circles of equal area and depth were placed inside and outside the envelope-detected photoacoustic target. For the simulated, experimental, and in vivo datasets described in Section III-A1, the ROI diameters were 6, 4, and 3 mm, respectively, with the center of the background ROI laterally shifted by 10, 8.5, and 10 mm, respectively, from the center of the target ROI. For the phantom and ex vivo datasets described in Section III-A2, the ROI diameters were 1.6 and 0.8 mm, respectively, with the center of the background ROI laterally shifted 5 mm from the center of the target ROI. In each dataset, the diameter of the ROIs was selected to match the axial dimension of the photoacoustic target being imaged, while the lateral shift between the ROIs was selected to accommodate deviations in the target shape from the ideal circle while minimizing the distance between the ROIs.

To demonstrate relationships between nodes in the first and second layers of FIG. 8, the datasets described in Sections III-A1 and III-A2 were employed. For each image in these datasets, the target shape and scale parameters, k and θ, respectively, were estimated within the target ROI of the envelope detected data, using closed-form expressions for estimating the parameters of a gamma distribution from random samples derived by Ye and Chen [43]. The mean background power (i.e., u) was estimated as the mean of the squares of the samples in the background ROI of the envelope detected data. The simulated, experimental, and in vivo datasets described in Section III-A1 were utilized to quantify the relationship between μ and channel SNR, which is included in the receiver characteristics node in FIG. 8. The phantom and ex vivo datasets described in Section III-A2 were utilized to quantify relationships between laser energy and the target parameters k and θ. A quadratic fit was implemented to describe the relationship between θ and laser energy in each imaging environment. The R² and root-mean-square error (RMSE) values were computed between the quadratic curve and the mean value of θ for each laser energy.

To demonstrate relationships between gCNR and the pre-ceding layers in FIG. 8, there are two possible options. First, the nodes in the second layer (i.e., target and background parameters) and the gCNR metric in the third layer can be connected with values of k, 0, and u measured from experimental data, by inserting these parameters into the mathematical expressions derived in Sections II-B4 and II-B5 to predict gCNR. This method was used to predict gCNR of the data described in Sections III-A1 and III-A2. This method was additionally leveraged to predict gCNR limits as a function of θ for minimum and maximum values of k and μ. Alternatively, to demonstrate the impact of system parameters in the first layer of FIG. 8 (e.g., channel SNR and laser energy) on the gCNR metric located in the third layer of FIG. 8, these indirect relationships were empirically characterized for different imaging environments using gCNR measurements.

To measure gCNR and validate gCNR predictions, true gCNR values were approximated using the histogram approach described by others. Histograms of the target and background signal powers within ROIs located in normalized DAS beamformed images were created, with bin widths computed as described elsewhere (see Appendix A for justification).

The histogram-based gCNR was then measured

$${gCNR}_{meas} = 1 - {\sum }_{k = 0}^{{N}_{b} - 1} min \{ {h}_{i} ( {x}_{k} ) , {h}_{o} ( {x}_{k} ) \} \qquad ( 29 )$$

where xₖ is the mean value of kth bin constructed from the input target and background signal power values, N_{b} is the number of bins of the histogram computed using a data-based method described elsewhere, and hᵢ and hₒ are the histogram values for the target and background, respectively.

To demonstrate the necessity of a photoacoustic-specific gCNR theory (rather than relying on the existing ultrasound theory for our proposed framework), gCNRₘₑₐₛ was directly compared with both the theory-based gCNR predictions for photoacoustic images presented in Section II-B5 and the ultrasound framework described in [24] (defined as gCNR_{US} in this article), using the datasets described in Sections III-A1 and III-A2. The resulting errors, eUS and ePA, were computed as follows:

$${e}_{US} = {gCNR}_{meas} - {gCNR}_{US} \qquad ( 30 )$$

$${e}_{PA} = {gCNR}_{meas} - {gCNR}_{PA} \qquad ( 31 )$$

where gCNR_{PA} represents gCNR_{PA1}, gCNR_{PA2}, or gCNR_{PA3}, depending on the case being evaluated. The mean absolute errors (MAEs), 11gUS and 11gPA, were then computed over M photoacoustic images in each dataset

$$Δ {g}_{US} = \frac{1}{M} {\sum }_{i = 1}^{M} \vert {e}_{US} [ i ] \vert \qquad ( 32 )$$

$$Δ {g}_{PA} = \frac{1}{M} {\sum }_{i = 1}^{M} \vert {e}_{PA} [ i ] \vert \qquad ( 33 )$$

where i represents the ith photoacoustic image in the dataset. The percent improvement, Δg, introduced by the photoacoustic framework was reported for each dataset based on the following equation:

$$Δ g = \frac{Δ {g}_{US} - Δ {g}_{PA}}{Δ {g}_{US}} \times 100 \% . \qquad ( 34 )$$

To demonstrate relationships between nodes in the third layer of FIG. 8 (i.e., SNR, CNR, and gCNR) and the performance of the computer vision-based target segmentation task (i.e., represented by the node in the fourth layer), each image in the phantom and in vivo datasets described in Section III-A3 was utilized. The target segmentation component of our photoacoustic-based visual servoing system was executed on the sequence of images in each dataset. To provide a brief summary of this previously published algorithm, each beamformed image was thresholded, dilated, and eroded. The segmentation algorithm then attempted to locate the fiber tip in each resulting image. If the algorithm successfully segmented the fiber tip in five consecutive beamformed image frames, then a further consistency check was used to prevent reflection artifacts from being misclassified as sources. The segmentation algorithm succeeded on a given image if the fiber tip was successfully located in the image and the output passed the consistency check when applicable.

The centroid of the region output by the segmentation algorithm was used as the center of the target ROI in each image. The ROI diameters were 3 mm in both datasets to match the axial dimensions of the target being imaged. The center of background ROIs was laterally shifted 10 mm from the center of the target ROIs to accommodate deviations in the shape of the segmented targets from the ideal circle. These ROIs were then used to measure gCNR, SNR, and CNR.

While there are multiple definitions of photoacoustic SNR and CNR, we use the following definitions to maintain consistency with our previous work:

$$SNR = \frac{{μ}_{t}}{{σ}_{b}} \qquad ( 35 )$$

$$CNR = \frac{\vert {μ}_{t} - {μ}_{b} \vert}{\sqrt{{σ}_{t}^{2} + {σ}_{b}^{2}}} \qquad ( 36 )$$

where μt and μb are the mean amplitudes of the target and background ROIs, respectively, within envelope detected DAS images, and σt and σb are the standard deviations of the amplitudes of the same data within the same target and background ROIs, respectively.

To characterize the relationship between segmentation accuracy and measured image quality metrics, a sigmoid curve was fit to datapoints from the phantom dataset described in Section III-A3. The associated R² and RMSE were computed to quantify the accuracy of the fit. To quantify accuracy variations across imaging environments, R² and RMSE values were also computed between the sigmoid fits and datapoints from the in vivo dataset described in Section III-A3. To demonstrate the effect of small changes in gCNR, SNR, and CNR on segmentation accuracy, the peak rate of increase of the sigmoidal fit was reported for each image quality metric.

To demonstrate the impact of thresholding on the target segmentation algorithm, each photoacoustic image from the phantom and in vivo datasets acquired in Section III-A3 was utilized. An amplitude-based threshold to was applied to each envelope detected DAS image in the range 0-1, corresponding to no (t₀=0) and all (to =1) pixels removed. In each thresholded image, the previously described automated ROIs were used to measure gCNR, SNR, and CNR using (29), (35), and (36), respectively. The minimum and maximum values of the measured image quality metrics were reported for each dataset as functions of t₀. The SNR and CNR metrics are expected to yield nonfinite values in thresholded images when σ_{b} in (35) or σₜ and σ_{b} in (36) are zero. To compare the stability of these image quality metrics, the percentage of gCNR, SNR, and CNR measurements that produced finite values were measured.

### IV. Results

### A. Background Power and Channel SNR Relationship

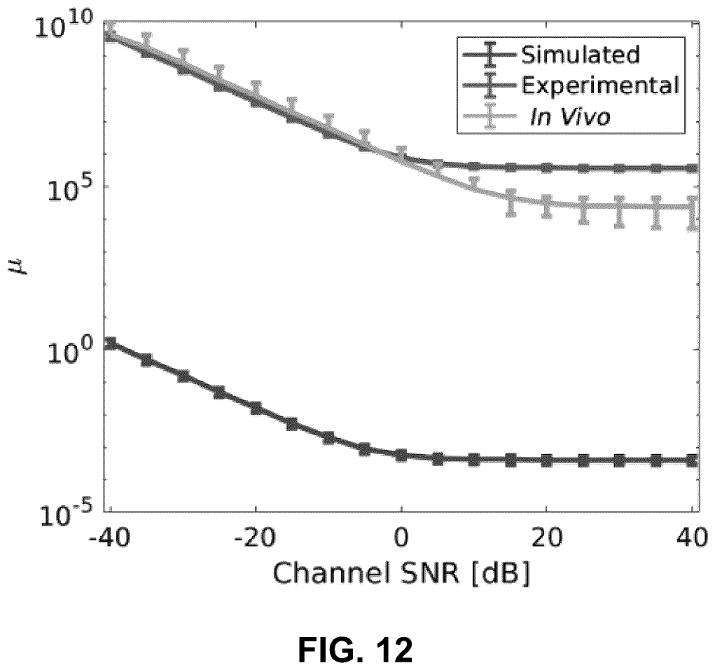

FIG. 12 shows the mean background power distribution u as a function of the channel SNR for the three datasets described in Section III-A1. Initially, u decreases as channel SNR increases, until an asymptote is reached. This initial relationship between μ and channel SNR can be explained by substituting (28) into (7) to obtain the following expression for u as a function of the desired channel SNR:

$$μ = {N}^{2} 〈 {μ}_{ch}^{2} 〉 \times {10}^{- \frac{{SNR}_{ch}}{10}} \qquad ( 37 )$$

where N is the number of receive channels. Taking the logarithm of both sides of (37) yields

$$log ( μ ) = log ( {N}^{2} ) + log ( 〈 {μ}_{ch}^{2} 〉 ) - \frac{{SNR}_{ch}}{10} \qquad ( 38 )$$

which matches the linear trend observed in the logarithmic plot of μ as a function of channel SNR in FIG. 12 for lower channel SNR values. The asymptotes of μ are 6.7×10⁻⁴, 3.0×10⁶, and 3.3×10³ for the simulated, experimental, and in vivo cases, respectively. These values correspond to the inherent noise in the raw channel data, which is significantly larger than the variance σ² of the added noise at higher channel SNR levels. This result is expected to produce gCNR values that remain fairly constant at these higher values of channel SNR (i.e., low noise and high signal conditions). In addition, the results in FIG. 12 provide an empirical demonstration of the edge connecting the receiver characteristics node to the background power distribution node in FIG. 8.

### B. Target Power and Laser Energy Relationship

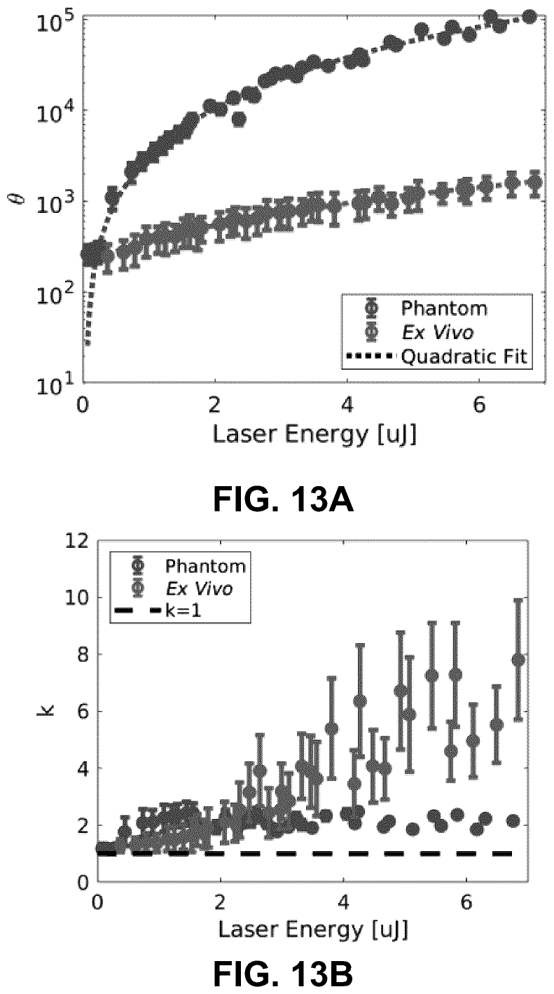

FIG. 13 shows measured values of the target parameters θ and k as functions of the laser energy of photoacoustic images acquired during the experiments described in Section III-A2. The parameter θ generally increases as laser energy increases. FIG. 13(a) shows quadratic curves fit to the mean values of θ with the phantom (R²=0.97 and RMSE=5.53×10³) and ex vivo tissue (R²=0.99 and RMSE=45.0) represented by the blue and orange dotted lines, respectively. The parameter k generally increases with laser energy in the ex vivo dataset. In the phantom dataset, k initially increases at lower laser energies and then remains low as the laser energy increases. These results provide an empirical demonstration of the edge connecting the laser parameters node to the target power distribution node in FIG. 8.

As shown in FIG. 8, the background parameter u does not depend on laser energy. However, as noted in Sections II-B2 and II-B4, the decision boundaries for gCNR predictions additionally depend on μ. Therefore, the range of possible gCNR predictions depends on the measured values of θ, k, and μ, which are reported in Table I, along with measured gCNR values for completeness. These values span Cases 1-4 of Section II-B4, and they represent the range of k and u values that can be used to predict gCNR as a function of θ and thereby predict the gCNR limits of these datasets.

TABLE 1
RANGE OF θ, k, μ, AND GCNR VALUES MEASURED FROM
THE PHANTOM AND Ex Vivo DATASETS PLOTTED IN FIG. 6

| | Phantom | Ex Vivo |
|---|---|---|
| θ | 1.5 × 10²-2.9 × 10⁵ | 1.1 × 10²-5.8 × 10³ |
| k | 0.69-4.23 | 0.67-16.33 |
| μ | 162.05-1182.11 | 92.58-745.68 |
| gCNR | 0.05-0.98 | 0.03-1.00 |

C. Target Power, Background Power, and gCNR

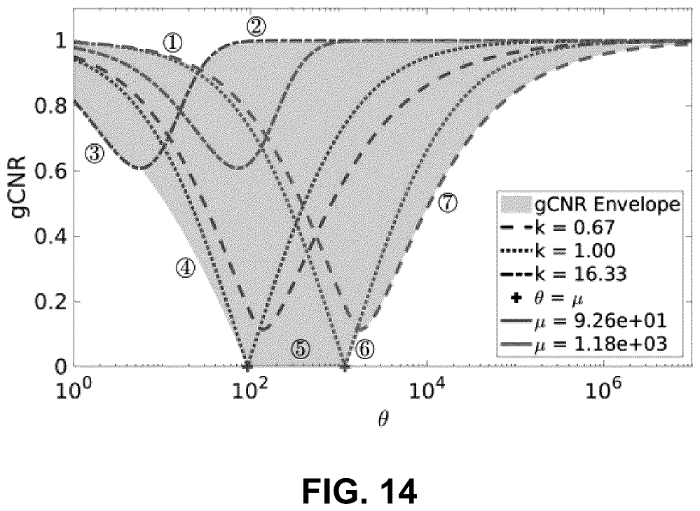

FIG. 14 shows the predicted limits of gCNR as a function of θ for independent selections of k and μ obtained from Table I. The upper bound of FIG. 14 can be divided into Regions 1 and 2, marked by the circled numbers, with corresponding values of θ, k, and μ reported in Table II. The values of μ and k in Regions 1 and 2 correspond to the maximum and minimum values of (u/k), respectively, within the selected datasets. The gCNR predictions saturate at unity when θ>>μ/k.

TABLE II
REGIONS FORMING THE UPPER AND LOWER BOUNDS
OF THE GCNR ENVELOPE IN FIG. 7

| | θₘᵢₙ | θₘₐₓ | k | μ |
|---|---|---|---|---|
| Region 1 | 1.0 | 27.13 | 0.67 | 1.18 × 10³ |
| Region 2 | 27.13 | 1.0 × 10⁷ | 16.33 | 92.6 |
| Region 3 | 1.0 | 5.67 | 16.33 | 92.6 |
| Region 4 | 5.67 | 92.6 | $\frac{μ}{θ}$ | 92.6 |
| Region 5 | 92.6 | 1.18 × 10³ | 1.0 | θ |
| Region 6 | 1.18 × 10³ | 1.76 × 10³ | $\frac{μ}{θ}$ | 1.18 × 10³ |
| Region 7 | 1.76 × 10³ | 1.0 × 10⁷ | 0.67 | 1.18 × 10³ |

The lower bound of the range of predicted gCNR values consists of five distinct regions, marked by the circled numbers 3-7 in FIG. 14. In Regions 3 and 7, the minimum possible gCNR either decreases or increases with an increase in θ. For Regions 4-6, the minimum possible gCNRs correspond to values of k that satisfy μ^{x}=μ. Therefore, k=μ/θ when the target power is gamma distributed in Regions 4 and 6. In Region 5, where the minimum possible gCNR is zero, the target power is exponentially distributed (i.e., k=1), and μ=0. These five regions indicate that it is sufficient to require kθ=μ to predict a minimum gCNR. If corresponding values of k and μ are not available (as in Regions 3 and 7), then the limiting values of k and μ may be selected to maximize the overlap between the target and back-ground power distributions. In addition to fulfilling the primary purpose of establishing expectations for photoacoustic gCNR across a range of target and background parameters, FIG. 14 also provides a theory-based demonstration of relationships between the target and background distribution nodes and the gCNR node in FIG. 8.

D. gCNR Predictions and Indirect Relationships

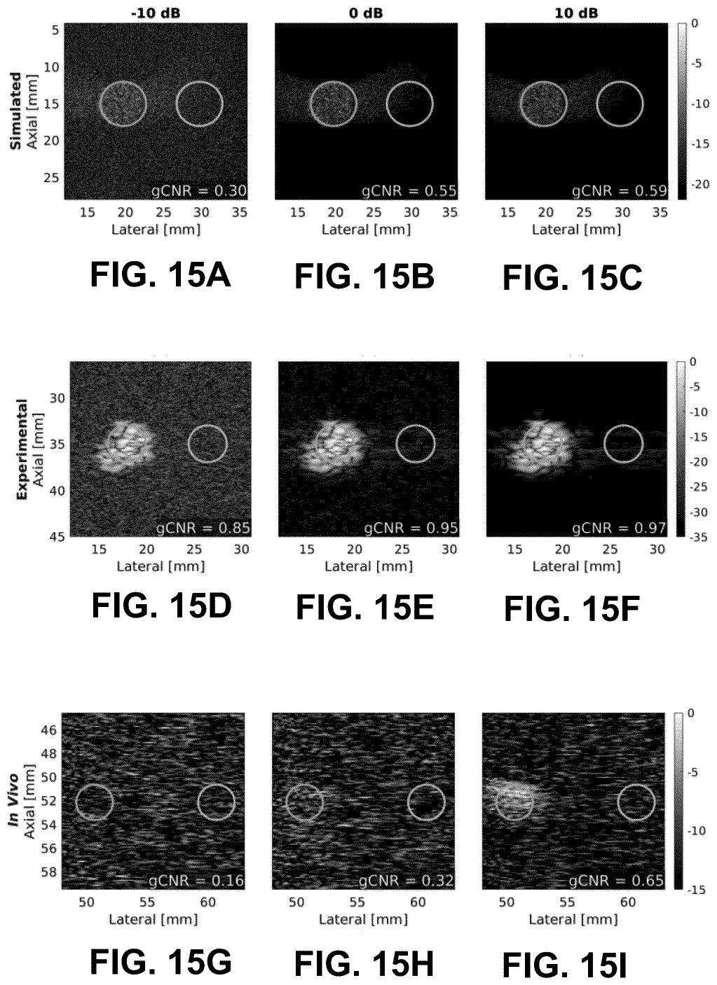

1) Channel SNR and gCNR: FIG. 15 compares DAS beam-formed images of simulated (6-mm-diameter target), experimental (5-mm-diameter target), and in vivo (2.5-mm-diameter target) data for channel SNR values of −10, 0, and 10 dB from the datasets described in Section III-A1. FIG. 15(a) shows a target which is not easily distinguishable from the background. This observation is confirmed by the low gCNR value of 0.30 for the simulated target at a channel SNR of −10 dB. Visibility improvements were obtained when the channel SNR value increased to 0 dB [FIG. 15(b)] and 10 dB [FIG. 15(c)]. FIG. 15(d) shows an image obtained in the water bath environment. Even at a low channel SNR value of −10 dB, the target was clearly visible, with a gCNR measurement of 0.85. When increasing the channel SNR value to 0 dB [FIG. 15(e)] and 10 dB [FIG. 15(f)], the target was more distinguishable from the background, with gCNR values of 0.95 and 0.97, respectively. FIG. 15(g) and(h), obtained by placing a catheter in an in vivo porcine heart, show targets that were difficult to detect with −10 and 0 dB channel SNR, respectively. At the higher channel SNR of 10 dB [FIG. 15(i)], the photoacoustic signal was more distinguishable from the background.

The results in FIG. 15 point to the indirect, qualitative relationship between channel SNR and gCNR. Target detectability improved as the channel SNR increased, although the gCNR improvement was not the same for each scenario. In addition, the target detectability was influenced by factors, such as the presence of side lobes and sound attenuation, even at higher channel SNR values [e.g., 10 dB in FIG. 15(c)]. As a result, images with the same channel SNR value (e.g., −10 dB) exhibited large variations in target visibility. These variations were quantified by the corresponding large variations in gCNR measured on the images [i.e., 0.30, 0.85, and 0.16 for FIG. 15(a), (d), and(g), respectively].

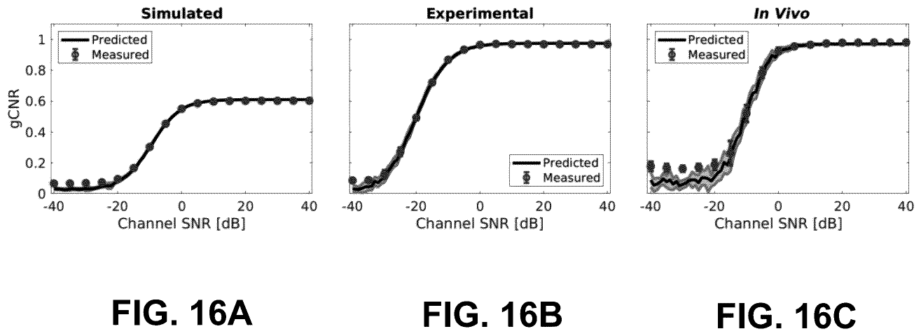

FIG. 16 quantifies measured and predicted relationships between gCNR and channel SNR for the simulated, experimental and in vivo photoacoustic image data described in Section III-A1. There is a clear sigmoidal relationship between gCNR and channel SNR, with a low-gCNR region for lower channel SNR values, a linear region, and an asymptote at higher channel SNR values. While there is generally good agreement between the predictions and measurements, the increased difference between predicted and measured gCNR at channel SNRs lower than-20 dB can be explained by modeling the target as a gamma-distributed signal. At lower channel SNR values, the amplitude of the noise in the tar-get ROI was sufficiently large to cause inaccuracies in the model of the target. These inaccuracies manifest as errors in the computation of the parameters k and θ from (2), and subsequently in the predicted gCNR values. Despite these inaccuracies, the gCNR predictions performed well even at low channel SNR values, with a peak mean absolute error of 0.04 for noisy in vivo images with channel SNR values ranging −40 to −20 dB. Overall, the results in FIGS. 15 and 16 provide empirical demonstrations of the indirect relationship between channel SNR and gCNR, which can be described by a combination of the edges along the directed path from the receiver characteristics node to the background power distribution node to the gCNR node in FIG. 8.

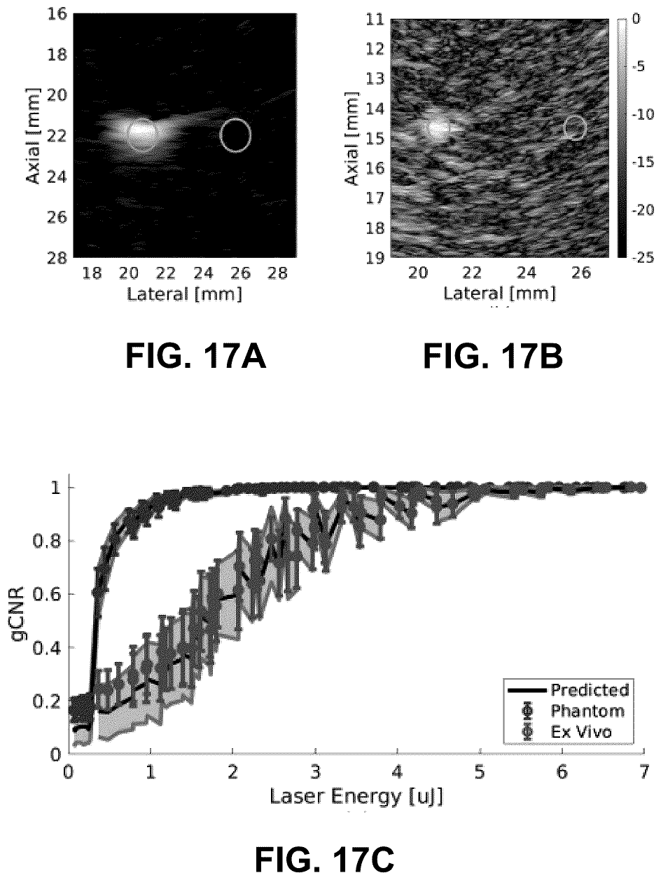

2) Laser Energy and gCNR: FIG. 17 shows DAS beam-formed photoacoustic images of the 2-mm-diameter optical fiber bundle in the plastisol phantom [FIG. 17(a)] and ex vivo caprine heart [FIG. 17(b)], taken from the datasets described in Section III-A2. FIG. 17(c) shows the predicted and measured gCNR as a function of laser energy for these datasets, demonstrating an increase in gCNR as energy increases. The asymptote of each curve also highlights that there is a limit to improvements in target detectability that can be achieved by increasing the laser energy beyond a threshold. This laser energy threshold depends on the properties of the acoustic environment (e.g., sound attenuation and acoustic clutter). The results in FIG. 17 provide empirical demonstrations of the indirect relationship between laser energy and gCNR, which can be described as a combination of the edges along the directed path from the laser parameters node to the target power distribution node to the gCNR node in FIG. 8.

TABLE III
MAES BETWEEN PREDICTED AND MEASURED
GCNR VALUES AND CORRESPONDING IMPROVEMENTS
WITH THE PROPOSED FRAMEWORK

| | Δg_{US} | Δg_{PA} | Improvement |
|---|---|---|---|
| | (US Framework [24]) | (PA Framework) | Δg (%) |
| Simulation | 2.6 × 10⁻² | 3.2 × 10⁻³ | 87.69% |
| Experimental | 5.9 × 10⁻³ | 3.7 × 10⁻³ | 37.58% |
| In Vivo | 8.4 × 10⁻² | 2.3 × 10⁻² | 72.13% |
| Phantom | 4.7 × 10⁻² | 1.3 × 10⁻² | 73.40% |
| Ex Vivo | 1.4 × 10⁻¹ | 2.0 × 10⁻² | 85.79% |

### E. Necessity of a Photoacoustic-Specific Framework

Table III reports the values of 11 gUS, 11 gPA, and 11 g for the results presented in FIGS. 16 and 17(c). These MAEs were computed for a total of 8100 images per simulated, experimental, or in vivo dataset described in Section III-A1, 9894 images in the phantom dataset described in Section III-A2, and 8800 images in the ex vivo dataset described in Section III-A2. The photoacoustic gCNR predictions have excellent agreement with the gCNR measurements in each dataset, with MAEs ranging 3.2 10⁻³−2.0×10⁻². In addition, our predictions outperformed the ultrasound-based framework with improvements ranging 37.58%-87.69% across the five datasets. More details about this comparison are available in Appendix B.

TABLE IV
CHARACTERIZATION OF THE SIGMOIDAL FITS
FOR PHANTOM AND In Vivo DATA IN FIG. 11

| Metric | Dataset | R² | RMSE | Peak Rate of Increase |
|---|---|---|---|---|
| gCNR | Phantom | 0.994 | 8.2 × 10⁻⁴ | 0.06 |
| | In Vivo | 0.907 | 1.2 × 10⁻² | |
| SNR | Phantom | 1.000 | 0.0 | 0.26 |
| | In Vivo | 0.976 | 2.0 × 10⁻⁷ | |
| CNR | Phantom | 0.997 | 5.16 × 10⁻⁴ | 0.07 |
| | In Vivo | 0.928 | 7.4 × 10⁻³ | |

F. Target Segmentation and gCNR Relationship

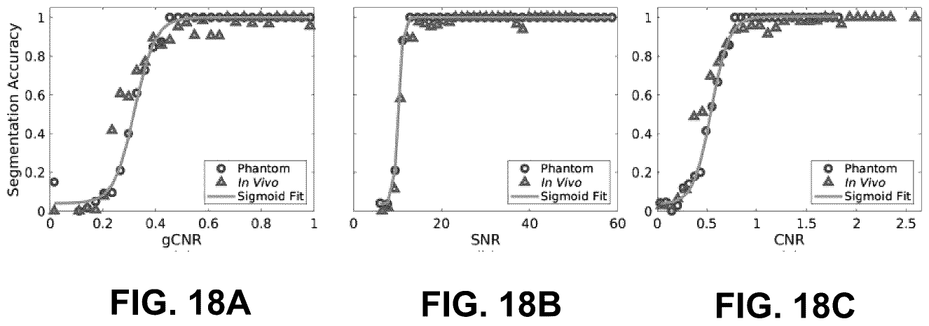

FIG. 18 shows the accuracy of the target segmentation algorithm as functions of gCNR, SNR, and CNR. The range of gCNR remains the same for both the phantom and ex vivo datasets in FIG. 18(a), as expected, because gCNR is bounded to the range [0, 1]. In contrast, SNR values in FIG. 18(b) range 5.2-59.5 dB for the phantom dataset and 5.8-50.2 dB for the ex vivo set. The CNR values also vary across datasets, ranging 0-1.8 and 0-2.6 for the phantom and ex vivo cases, respectively. These variations in the ranges of SNR and CNR demonstrate the sensitivity of these metrics to multiple nodes in FIG. 8 when compared with gCNR measurements.

FIG. 18 also shows sigmoidal curves fit to the segmentation accuracy as a function of gCNR, SNR, and CNR using the phantom dataset described in Section III-A3. Table IV lists the R² and RMSE between each fit and the corresponding phantom or in vivo datapoints. The SNR results have the highest R2 value and lowest RMSE due to the presence of fewer data points in the linear region of the sigmoid fit. The gCNR and CNR results have similar R² and RMSE values. The last column of Table IV reports the maximum rate of increase in segmentation accuracy as a function of the corresponding image quality metric. This peak rate of increase demonstrates that gCNR and CNR are more sensitive than SNR to detect small changes in segmentation accuracy. Note that a threshold of t₀=0.7 was applied to improve the accuracy of the segmentation algorithm implemented in FIG. 18, while no thresholds were applied to report the corresponding image quality metrics.

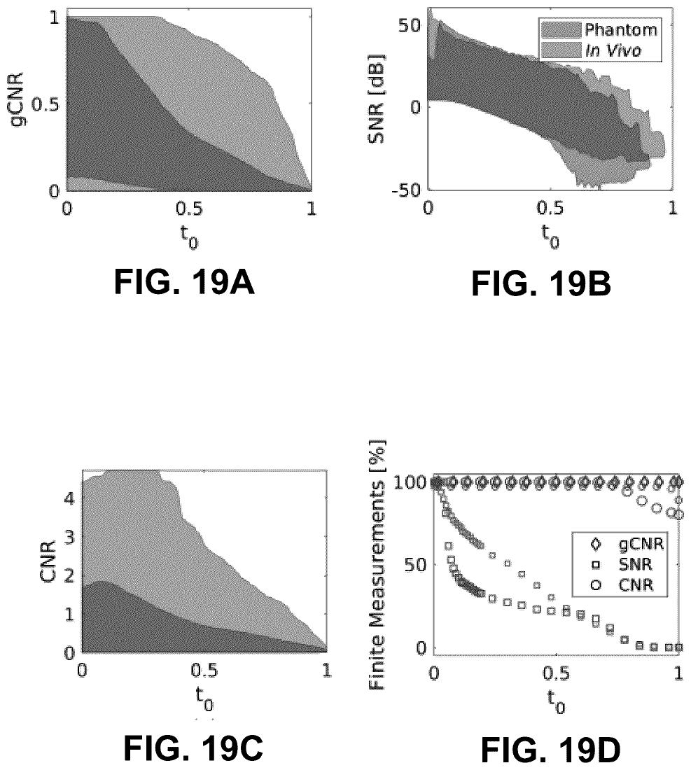

FIG. 19(a)-(c) shows the minimum and maximum values of gCNR, SNR, and CNR measurements, respectively, as functions of t₀. Of these three metrics, only gCNR monotonically decreases with an increase in t₀ in both the phantom and in vivo datasets, which agrees with the expectations concluded in Section II-C. In comparison, the CNR measurements achieve peaks of 1.8 and 4.7 at threshold values t₀=0.08 and t₀=0.11 in the phantom and in vivo datasets, respectively. The absence of a monotonic decrease is similarly observed with SNR when t₀>0.5 in FIG. 19(b).

FIG. 19(d) shows the percentage of gCNR, SNR, and CNR measurements yielding finite values as a function of to in the phantom and in vivo datasets. The gCNR measurements remain finite across 100% of the images in both datasets, which is expected because this metric will always produce a value that is bounded by [0, 1]. In comparison, the percentage of finite CNR measurements reduces from 97.1% at t₀=0 to 91.0% at t₀=0.82. More substantial decreases in the percentage of finite measurements are observed in the SNR metric, reducing from 100% and 97.1% at t₀=0 in the phantom and in vivo datasets, respectively, to 41.9% and 73.7%, respectively, at t₀=0.1. These results demonstrate the sensitivity of SNR and CNR measurements to thresholding and the robustness of gCNR to the same image manipulation.

### V. Discussion

A. Relationships Elucidated by Photoacoustic gCNR Theory

Given the desired photoacoustic system parameters and the associated target and background distribution parameters, we demonstrated that we can successfully predict gCNR, which may then be used to determine expected segmentation accuracy for visual servoing tasks. Although results are demonstrated in the context of photoacoustic target segmentation, our overall framework is more generally applicable to a variety of related computer vision-based tasks. We used this framework to demonstrate direct and indirect relationships among system parameters, target and background distribution parameters, and image quality metrics, such as gCNR. In addition, the directed graph representation supports the description of indirect relationships in the context of specific direct relationships.

There are a total of seven direct and two indirect relationships among system parameters, power distribution para-meters, image quality metrics, and computer vision-based task performance identified by this framework. The direct relationship between channel SNR and μ (FIG. 12) informs the indirect relationship between channel SNR and gCNR. In particular, there is a sigmoidal relationship between gCNR and channel SNR (FIG. 16), which contains toe, linear, and shoulder regions. When comparing these regions with FIG. 12, it is apparent that the toe and linear regions of FIG. 16 correspond to the linear region of FIG. 12. In the toe region of FIG. 16, μ is sufficiently large such that the target region is overwritten by noise, resulting in consistently low gCNR. As μ decreases in FIG. 12, the corresponding linear region of FIG. 16 demonstrates an increase in gCNR. The shoulder region of FIG. 16 corresponds to the asymptote in FIG. 12, where the corresponding channel SNR values are sufficiently large and the raw channel data remains relatively unchanged after the addition of noise, resulting in constant μ and gCNR values as channel SNR further increases.

Similarly, the direct relationship between laser energy and the target power distribution parameters (FIG. 13) informs the indirect relationship between laser energy and gCNR (FIG. 17). In particular, based on (3), an increase in θ or k translates to an increase in μ^{x}. These increases further translate to increased separation between the target and background distributions and improved gCNRs (assuming no significant change in μ). The larger increase in θ compared with k for the phantom data in FIG. 13 manifests as a large increase and eventual saturation of gCNR to 0.98 in FIG. 17(c). For laser energy levels ranging 4-7 μJ in FIG. 13(a), although the measured θ values are smaller in the ex vivo dataset compared with the phantom dataset, these same datasets have similarly high gCNR values over the same energy range in FIG. 17(c). This similarity can be explained by the higher values of k for the ex vivo dataset shown in FIG. 13(b) over the same range of laser energies. These observations demonstrate relationships between the target parameters and the imaging environment (e.g., phantom and ex vivo tissue) and between the target parameters and the laser parameters (e.g., laser energy), which are both represented in FIG. 8.

In addition, Regions 1-7 in FIG. 14 describe the limits of relationships among k, θ, μ, and gCNR. These regions indicate that the range of achievable gCNR can be completely described for any imaging system and environment using descriptions of target and background parameters. In particular, the presence or absence of each region solely depends on the relative values of k, θ, and μ. Therefore, the achievable ranges of gCNR can be analyzed independently of the underlying parameters of the photoacoustic imaging system (e.g., target size, laser energy, and imaging environment), and these ranges can be compared across different photoacoustic imaging systems.

### B. Impact of Proposed Framework

The proposed framework demonstrates the feasibility of predicting the performance of a computer vision task using measurements of image quality metrics. The results in FIGS. 18 and 19 and Table IV highlight three advantages of gCNR over SNR and CNR when predicting performance of the target segmentation algorithm applied to phantom and in vivo datasets. First, segmentation accuracy is more robust to small changes in gCNR compared with SNR (FIG. 18 and Table IV). Second, the gCNR metric has finite upper and lower bounds, whereas SNR and CNR are unbounded (FIG. 18). Therefore, gCNR measurements are easier to interpret than SNR and CNR in the context of computer vision tasks, such as target segmentation, and they do not experience the data dropout observed in FIG. 19(d) as threshold values increase. Third, SNR and CNR may be arbitrarily increased through image manipulation techniques, such as thresholding, while gCNR experiences a monotonic decrease with increasing threshold values, as observed in FIG. 19(a)-(c). In addition, our previous work [22] demonstrated the large variations in SNR and CNR values for small increases in gCNR in simulated, phantom, and in vivo photoacoustic images. Considering that the sigmoidal relationship between segmentation accuracy and gCNR was successfully characterized with phantom data, the expected performance of a computer vision-based system in a surgical setting can be predicted using this type of phantom-based characterization.

Overall, the presented framework has the potential to improve the design of photoacoustic imaging systems by addressing the challenge of achieving a desired gCNR in beamformed images. The associated relationships identified and characterized in this article improve the ability of design engineers to optimize parameters, such as laser energy and transducer characteristics when designing systems for specific computer vision tasks. The gCNR metric has the potential to be incorporated into the design process of photoacoustic imaging systems to resize system components and reduce costs, potentially enabling the proliferation of smaller photoacoustic imaging systems better suited for surgical and interventional suites. Thus, employing gCNR as a prediction tool has the potential to improve the performance of tasks such as tracking catheter tips during cardiac catheterization procedures [18], placing pedicle screws during spinal fusion surgeries, visualizing the ureters, uterine arteries, and laparoscopic tool tips during hysterectomies, and visual servoing features of interest in a photoacoustic image.

The framework presented in FIG. 8 additionally promises to improve the efficiency of preoperative tasks for photoacoustic-guided surgical and interventional procedures. For example, previous work to improve cardiac catheterization procedures required multiple trial and error attempts to determine the optimal laser energy for successful visual servoing of photoacoustic images. Characterizing a visual servoing system with respect to gCNR and the underlying target and background statistics enables the incorporation of a theory-based approach to selecting desired laser energy levels and reduces dependency on trial and error.

In particular, a photoacoustic image could be acquired once at low laser energy prior to a surgical procedure to measure gCNR. Using the relationship between gCNR and laser energy measured from an ex vivo setup similar to the surgical environment [e.g., FIG. 17(c)] and the relationship between the segmentation accuracy and gCNR established in FIG. 18(a), the gCNR prediction framework would provide the increase in laser energy required to achieve a desired segmentation accuracy range (e.g., 80%-100%). Acquiring a photoacoustic image at the updated laser energy would enable measurements of gCNR (from the acquired image), predictions of the segmentation accuracy [based on the sigmoidal fit in FIG. 18(a)], and confirmation that the visual servoing process will be conducted within the desired segmentation accuracy range. Based on the selected segmentation accuracy range, this theory-based process inherently allows us to balance the tradeoff between minimizing laser energy and maximizing target segmentation accuracy

### C. Limitations and Future Work

One limitation of the presented gCNR predictions is their dependence on the PDF of the target and background signal powers. Factors such as the target size, target position, target shape, beamformer selection, and ROI placement cannot be explicitly modeled in these predictions, although we have observed that these factors influence gCNR measurements in experimental data. These and other factors (including the appearance of side lobes and background contributions that cannot be modeled by our gCNR predictions) are nonetheless hypothesized to contribute to the reported empirical observations. In addition, our ability to accurately estimate the parameters of the target and background power distributions depends on the selection of the corresponding ROIs in the photoacoustic image. However, the gCNR prediction framework shares this limitation with both the histogram-based gCNR measurement technique and traditional image quality metrics, such as SNR, CNR, and contrast, which are measured using ROIs.

Note that the model of the target power distribution presented in Section II-B2 was developed for photoacoustic images created with envelope detection. Without envelope detection, the target and background distributions have modified shapes, with increased probability densities observed at lower power values. If image formation is altered by removing envelope detection, these observed differences would require the derivation of a new power distribution model that will accurately produce lower gCNR predictions in the absence of envelope detection.

Future work includes extending the presented framework to other beamformers to demonstrate the improvements in target detectability achieved by different image formation processes. Previous work from our group empirically demonstrated improvements in gCNR measured from images recon-structed using SLSC beamforming compared with images reconstructed from the same raw channel data using DAS beamforming. However, a theoretical analysis of the impact of beamformers on target detectability would require specific models of the target and background power distributions for each beamformer being analyzed.

Another potential application of our framework is to estimate the parameters of a photoacoustic imaging system using the target and background distribution parameters obtained from a beamformed image. For example, channel SNR is traditionally utilized to estimate noise in photoacoustic images. However, in clinical environments, without access to channel data, it would not be possible to estimate the channel SNR that exists in photoacoustic images to help with troubleshooting steps needed for real-time improvement of poor image quality (e.g., increasing laser energy versus adjusting the dynamic range). Our group recently introduced a method to estimate channel SNR from gCNR without access to raw channel data. This approach can be incorporated into our frame-work to link the image domain represented by the second and third layers in FIG. 8 to the channel data domain represented by the first layer in FIG. 8. This link has the potential to improve the rapid decision-making that is often necessary in life or death situations, enhance the reproducibility of clinical imaging environments, and provide consistently high-quality photoacoustic image acquisitions.

In addition, the extension of this framework to raw channel data would enable performance benchmarking for a new class of deep learning-based photoacoustic visual servoing systems that segment photoacoustic sources from raw channel data. This framework may also be utilized to compare the performance of amplitude-based and coherence-based visual servoing systems.

### VI. Conclusion

This example is the first to present a novel framework to establish relationships among photoacoustic imaging system parameters, image quality, and computer vision-based task performance. This framework leveraged gCNR to quantify the relationships between system parameters (e.g., channel SNR, laser energy, and frame averaging) and photoacoustic image quality. A critical component of this framework involved predicting the gCNR of photoacoustic images using the statistics of the target and background signal powers. We presented a theoretical derivation of our gCNR predictions and validated these predictions on simulated, experimental, and in vivo data across multiple channel SNRs and laser energies. This framework additionally enabled characterizations of the relationships between system parameter settings and image characteristics, including gCNR as a function of laser energy, the variation of the target and background distribution parameters as functions of laser energy and channel SNR, and the dependence of gCNR on the relative values of the mean target and background powers. Finally, we leveraged our novel framework to quantify the accuracy of a photoacoustic target segmentation algorithm as a function of gCNR and demonstrate the robustness of gCNR to thresholding. The proposed framework has the potential to be extended to other computer vision-based tasks (e.g., target tracking and image classification) and to improve the process of designing photoacoustic imaging systems, specifically the selection of hardware components such as lasers and software components such as image quality improvement techniques.

### Fixed Versus Data-Based Histogram Bin Widths

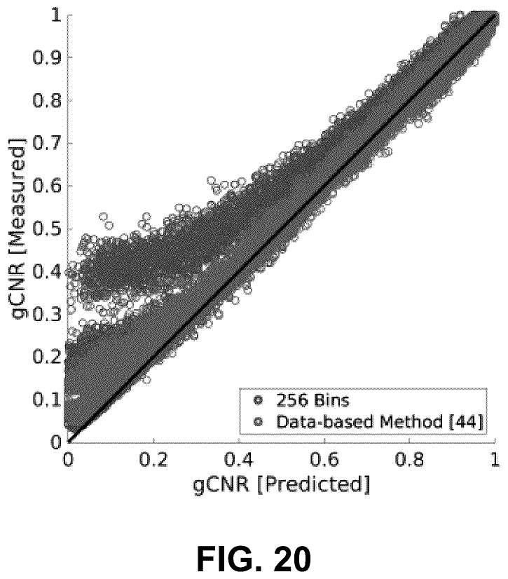

FIG. 20 compares histogram-based gCNR measurements using two different bin selection methods as a function of the values output by the gCNR prediction framework for photoacoustic images, for the five datasets described in Sections III-A1 and III-A2. Existing literature involving gCNR measurements uses a fixed number of bins to compute the histograms of both the target and background signal powers. However, statistics literature suggests that using a data-based bin width selection method would provide better results. In this example, we use a bin width selection method previously developed, where the bin width is a function of the number of samples and standard deviation of the target and background signal powers. FIG. 20 shows a strong agreement between the gCNR values measured using this data-based method, represented by the orange circles, and the gCNR predictions. In contrast, the gCNR values measured by fixing the number of bins to 256 deviate significantly from the predicted gCNR values, as shown by the blue circles in FIG. 20. These results suggest that the data-based method is superior to fixing the number of bins to 256. We expect similar results to be obtained with ultrasound images and the ultrasound framework previously published.

### Advantage of Photoacoustic Framework Over Ultrasound Framework for GCNR Predictions

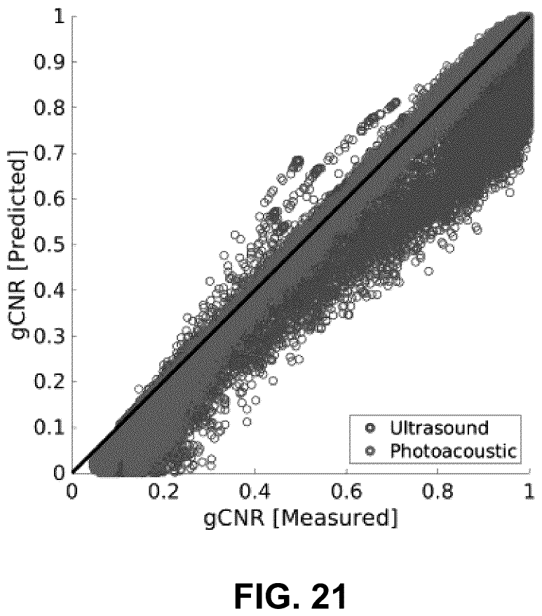

FIG. 21 shows a comparison of gCNR prediction frameworks for ultrasound and photoacoustic images as a function of gCNR measurements for the five datasets described in Sections III-A1 and III-A2. The gCNR measurements are obtained from normalized photoacoustic images with histogram bin widths selected using the data-based method previously described. The black line represents the ideal case where the gCNR predictions are exactly equal to the gCNR measurements. The gCNR predictions output by our framework for photoacoustic images are closer to the ideal black line than the gCNR values output by the ultrasound model previously published. This matches our expectation that photoacoustic images require a specific gCNR prediction framework based on the differences in target power statistics between ultrasound and photoacoustic images. The gCNR values predicted with the ultrasound and photoacoustic frameworks are more similar as gCNR decreases because the exponential distribution is sufficient to model the target power at low gCNR values in both cases. However, the observed deviation from the ideal line at these lower gCNR values is likely due to measurement inaccuracies. In particular, the histogram-based method seems to inaccurately represent the target and background distributions at low gCNR values.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, and/or computer readable media or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A system, comprising:
an electromagnetic radiation source configured to produce electromagnetic waves;
an optical fiber operably connected to the electromagnetic radiation source, which optical fiber is configured to transmit the electromagnetic waves from the electromagnetic radiation source to one or more selected sites in and/or on a subject to generate acoustic waves at least proximal to the one or more selected sites in and/or on the subject;
a medical device operably connected to the optical fiber;
a robotic device comprising an acoustic sensor, which robotic device is configured to position the acoustic sensor to receive the acoustic waves; and,
a controller operably connected at least to the robotic device, which controller comprises a processor and a memory communicatively coupled to the processor, which memory stores instructions which, when executed on the processor, perform operations comprising:
positioning the acoustic sensor within sensory communication of the acoustic waves using the robotic device such that the acoustic sensor receives the acoustic waves to produce image data; and,
tracking a position of the medical device using the image data and one or more photoacoustic point source localization algorithms selected from the group consisting of: an amplitude-based algorithm, a coherence-based algorithm, and a deep learning-based target segmentation algorithm.

2. The system of claim 1, wherein the instructions comprise an electronic neural network and wherein the deep learning-based target segmentation algorithm is implemented using an electronic neural network.

3. The system of claim 1, wherein the instructions which, when executed on the processor, perform tracking the position of the medical device using the image data and two or more of the photoacoustic point source localization algorithms.

4. The system of claim 1, wherein the medical device comprises a needle, a catheter, or a surgical implement.

5. The system of claim 1, wherein the image data comprises beamformed image data and/or raw channel data.

6. The system of claim 1, wherein the subject comprises a human subject.

7. A method of tracking a position of a medical device, the method comprising:
moving an optical fiber that is operably connected to a medical device to one or more selected sites in and/or on a subject, which optical fiber is operably connected to an electromagnetic radiation source;
transmitting electromagnetic waves from the electromagnetic radiation source to the one or more selected sites in and/or on the subject through the optical fiber to generate acoustic waves at least proximal to the one or more selected sites in and/or on the subject;
positioning an acoustic sensor within sensory communication of the acoustic waves using a robotic device that is operably connected to the acoustic sensor such that the acoustic sensor receives the acoustic waves to produce image data; and,
tracking a position of the medical device using the image data and one or more photoacoustic point source localization algorithms selected from the group consisting of: an amplitude-based algorithm, a coherence-based algorithm, and a deep learning-based target segmentation algorithm.

8. The method of claim 7, wherein the deep learning-based target segmentation algorithm is implemented using an electronic neural network.

9. The method of claim 7, comprising tracking the position of the medical device using the image data and two or more of the photoacoustic point source localization algorithms.

10. The method of claim 7, wherein the medical device comprises a needle, a catheter, or a surgical implement.

11. The method of claim 7, wherein the image data comprises beamformed image data and/or raw channel data.

12. The method of claim 7, comprising tracking the position of the medical device in substantially real-time.

13. The method of claim 7, wherein the subject comprises a human subject.

14. A computer readable media comprising non-transitory computer executable instruction which, when executed by at least electronic processor, perform at least:
moving an optical fiber that is operably connected to a medical device to one or more selected sites in and/or on a subject, which optical fiber is operably connected to an electromagnetic radiation source;
transmitting electromagnetic waves from the electromagnetic radiation source to the one or more selected sites in and/or on the subject through the optical fiber to generate acoustic waves at least proximal to the one or more selected sites in and/or on the subject;
positioning an acoustic sensor within sensory communication of the acoustic waves using a robotic device that is operably connected to the acoustic sensor such that the acoustic sensor receives the acoustic waves to produce image data; and,
tracking a position of the medical device using the image data and one or more photoacoustic point source localization algorithms selected from the group consisting of: an amplitude-based algorithm, a coherence-based algorithm, and a deep learning-based target segmentation algorithm.

15. The computer readable media of claim 14, wherein the deep learning-based target segmentation algorithm is implemented using an electronic neural network.

16. The computer readable media of claim 14, wherein the non-transitory computer-executable instructions which, when executed by the electronic processor, further perform at least:
tracking the position of the medical device using the image data and two or more of the photoacoustic point source localization algorithms.

17. The computer readable media of claim 14, wherein the medical device comprises a needle, a catheter, or a surgical implement.

18. The computer readable media of claim 14, wherein the image data comprises beamformed image data and/or raw channel data.

19. The computer readable media of claim 14, wherein the non-transitory computer-executable instructions which, when executed by the electronic processor, further perform at least:
tracking the position of the medical device in substantially real-time.

20. The computer readable media of claim 14, wherein the subject comprises a human subject.
